(19) United States
(12) Patent Application Publication
(10) Pub. No.: US 2009/0221690 A1
(43) Pub. Date: Sep. 3, 2009

(54) Pharmaceutical Composition for the Prevention/Treatment of Bone Disorders and a Process for the Preparation Thereof

(75) Inventors: Rakesh Maurya, Uttar Pradesh (IN); Geetu Singh, Uttar Pradesh (IN); Pandruvada Subramanyam Narayan Murthy, Uttar Pradesh (IN); Sandhya Mehrotra, Uttar Pradesh (IN); Divya Singh, Uttar Pradesh (IN); Biju Bhargava, Uttar Pradesh (IN); Man Mohan Singh, Uttar Pradesh (IN)

Correspondence Address: ARENT FOX LLP, 1050 CONNECTICUT AVENUE, N.W., SUITE 400, WASHINGTON, DC, 20036, US

(73) Assignee: COUNCIL OF SCIENTIFIC AND INDUSTRIAL RESEARCH, NEW DELHI, IN (US)

(21) Appl. No.: 12/281,098

(22) PCT Filed: Feb. 27, 2007
(86) PCT No.: PCT/IB07/00468

§ 371 Date: May 1, 2009

(30) Foreign Application Priority Data
Feb. 28, 2006 (IN) 0522/DEL/2006

(51) Int. Cl. A61K 31/352; C07D 311/04

(52) U.S. Cl. 514/453; 549/403

(57) ABSTRACT

Osteoporosis is one of the major problems in our aging society. Osteoporosis results in bone fracture in older members of the population, especially in post-menopausal women. In traditional medicine, there are many natural crude drugs that have the potential for use to treat bone diseases. So far, there is no report in literature on anti-osteoporosis (bone forming) activity of Butea species. It was thought to study the anti-osteoporotic activity of this plant. Thus, the present invention provides a pharmaceutical composition from the extracts of Butea monosperma for prevention or treatment of bone disorders, process of preparation and use thereof.

### FIELD OF THE INVENTION

The present invention relates to the field of pharmaceuticals and organic chemistry and provides new plant extracts, their fractions, subfractions, pure compounds isolated from these or other natural sources or synthesized, their pharmaceutically acceptable salts and compositions that are useful for the prevention or treatment of various medical indications associated with estrogen independent or dependent diseases or syndromes preferably in prevention or treatment of diseases and syndromes caused in humans and animals in particular:
a) Osteoporosis, bone loss, bone formation;
b) bone formation during Type-II/age related/senile osteoporosis, period of development and growth to attain higher peak bone mass, bone fracture healing, promotion of new bone formation in vitro I in vivo for replacement of defective bone;
c) estrogen related diseases or syndromes, preferably diseases or syndromes caused by an estrogen-deficient state in a mammal;
d) cardiovascular effects more particularly hyperlipidaemia, thrombosis and vasomotor system;
e) neurodegenerative effects such as stroke, senile dementia-Alzheimer type and Parkinson disease;
f) menopausal symptoms including hot flushes, urogenital atrophy, depression, mania, schizophrenia and the like, urinary incontinence, relief of dysmenorrhea; relief of dysfunctional uterine bleeding, an aid in ovarian development, treatment of acne and hirsutism;
g) cancers such as prostatic carcinoma, cancer of breast, cancer of uterus, cancer of the cervix and cancer of the colon;
h) control or regulation of fertility in humans and in other animals;
i) for use in the prevention of threatened or habitual abortion;
j) suppression of post-partum lactation;
k) physiological disorders such as obesity, depression etc.;

The present invention further relates to the processes for the preparation of pharmaceutically active extracts, fractions, subfractions, isolation of pure compounds, their pharmaceutically acceptable salts and compositions of the principal aspect of the present invention.

### BACKGROUND OF THE INVENTION

Osteoporosis, which has been defined as a “state of low bone mass”, is one of the major problems in our aging society. It is a disease characterized by micro architectural deterioration of bone tissue leading to enhanced bone fragility and consequent increase in fracture risk in older members of the population. It is known to affect >50% of women and 30% men over the age of 50 years. In women, there is also an accelerated rate of bone loss immediately and for variable number of years following menopause.

Efforts to reduce this risk factor and incidence of fractures have resulted in the development of compounds that conserve skeletal mass by inhibiting bone resorption and/or by enhancing bone formation (Dwivedi I, Ray S, 1995 “Recent developments in the chemotherapy of osteoporosis” Progress in Drug Research 45, 289-338, Editor E Jucker, Birkhauser Vela; Marshall D H, Horsmann A, Nordin B E C, 1977, “The prevention and management of post-menopausal osteoporosis” Acta Obstet Gynecol Scand (Suppl) 65:49-56; Hutchinson T A, Polansky S M, Feinstein A R, 1979, “Postmenopausal estrogen protect against fractures of hip and distal radius: A care-control study” Lancet 2:705-709. Estrogen replacement therapy also has positive effect on CVS & CNS related disorders (Lobo R A, 1990, “Cardiovascular implication of estrogen replacement therapy” Obstetrics & Gynaecology 75:185-245; Mendelson M E, Karas R H, 1994, “Estrogen and the blood vessel wall” Current opinion in Cardiology 1994:619-626; Stampfer M J, Colditz G A, 1991, “Estrogen replacement therapy and coronary heart disease: a quantitative assessment of the epidemiological evidence” Preventive Medicine 20:47-63).

Most of the pharmacological agents available for clinical use such as calcium, vitamin D and its analog, estrogen, calcitonin, bisphosphonates, raloxifene etc. act by decreasing the rate of bone resorption, thereby slowing the rate of bone loss. Timely administration of such antiresorptive agents prevents bone loss. However, bone once lost cannot be recovered by use of such antiresorptive agents.

In traditional medicine, there are many natural crude drugs that have the potential to treat bone diseases. However, not much laboratory work has been reported evaluating their possible development and use, except ipriflavone, a natural product derivative, which has been used clinically for this indication.

The natural products included in this patent have been demonstrated to promote proliferation and differentiation of osteoblasts, matrix maturation and mineralization in vitro in a number of assays and increase bone mineral density and bone mechanical strength following prolonged treatment in vivo and would be of tremendous use not only in fast fracture healing and management of age-related (Type-II) osteoporosis, but might also help in attaining higher peak bone mass when administered during the period of growth and development, promote new bone formation in vitro/in vivo for replacement of defective bone and prevention of resorption in estrogen deficiency states including post-menopausal osteoporosis. Currently the only agents reported to show bone formation activity include (a) parathyroid hormone, which is to be administered parenterally and increases bone resorption at higher doses, (b) fluoride, excessive intake of which is also known to cause osteoporosis and (c) androgens by virtue of their anabolic activity. This is the first agent of its kind from natural sources and would be developed as an oral formulation for human use and welfare. The following specification particularly describes and ascertains the nature of this invention and the manner in which it is to be performed:

Butea monosperma (Lam.) Taub (Syn. Butea frondosa; Family Fabaceae) popularly known as ‘dhak’ or ‘palas’. The species of Butea include Butea monosperma, Butea parviflora, Butea minor and Butea superba. These are widely distributed throughout India [The Wealth of India-Raw Materials. 341-346, 1988, PID, CSIR, New Delhi]. The plants of this genus are well known for their colouring matters.

The roots of Butea monosperma are useful in the treatment of night blindness and other eye diseases [Mengi, S. A., Deshpande, S. G. Journal of Pharmacy and Pharmacology 47, 997-1001, 1995]. It is reported to possess antifertility, aphrodisiac, analgesic and anthelmintic activities [The Wealth of India-Raw Materials, pp. 341-346, 1988, PID, CSIR, New Delhi]. The tubers of Butea superba have been found to contain estrogenic substances similar to follicle hormones [Schoeller, W., Dohrn, M., Hohlweg, W. Naturwissenschaften 28, 532-533,1940]. Roots of Butea superba show rejuvenating activity [Pangsrivongse, K. Rev. Filipina Med. Farm. 29, 12-14, 1938]. The root barks of Butea superba shows 65% inhibitory activity on acetylcholinesterase [Kornkanok, I., Prapapan, T., Kanchanaporn, C., Thitaree, Y., Warawit, T. Journal of Ethnopharmacology 89, 261-264, 2003]. The preparation of Butea superba tubers has been used as an alternative herbal treatment for erectile dysfunction in males [Cherdshewasart, W., Nimsakul, N. Asian Journal of Andrology 5, 243-246, 2003]. The stem bark of Butea monosperma displays antifungal activity, which is due to the presence of an active constituent (−)-medicarpin [Bandara, B. M., Kumar, N. S., Samaranayake, K. M. Journal of Ethnopharmacology 25, 73-75, 1989]. It has also been reported to possess aphrodisiac, anthelmintic antibacterial and antiasthmatic properties [The Wealth of India-Raw Materials pp. 341-346, 1988, PID, CSIR, New Delhi]. A flavonol glycoside isolated from the stem of Butea superba shows antimicrobial activity [Yadava, R. N., Reddy, K. I. Journal of Asian Nat. Prod. Res. 1, 139-145,1998].

The leaves of Butea monosperma exhibit ocular anti-inflammatory activity in rabbits [Mengi, S. A., Deshpande, S. G. Journal of Pharmacy and Pharmacology 47, 997-1001, 1995] and strong antimicrobial activity [Zaffar, R., Singh, P., Siddiqi, A. A. Indian J. Fores. 12, 328-329, 1989].

An extract from the flowers of Butea monosperma is used in India for the treatment of liver disorders and two antihepatotoxic flavonoids, isobutrin and butrin have been isolated from the extract [Wagner, H., Geyer, B., Fiebig, M., Kiso, Y., Hikino, H. Planta Medica 52, 77-79, 1986]. It shows anticonvulsive activity, due to the presence of a triterpene [Kasture, V. S., Kasture, S. B., Chopde, C. T. Pharmacology, Biochemistry and Behavior 72, 965-972, 2002]. Alcoholic extract of flowers of Butea monosperma has also been reported to exhibit antiestrogenic [Shah, K. G., Baxi, A. J., Shukla, V. J., Dave, K. K., De, S., Ravishanker, B. Indian Journal of Pharmaceutical Sciences 52, 272-5, 1990; Laumas, K. R., Uniyal, J. P. Indian Journal of Experimental Biology 4, 246, 1966] and antifertility [Razdan, M. K., Kapila, K., Bhide, N. K. Indian Journal of Physiology and Pharmacology 14, 57-60, 1970] activities, Butin isolated from its flowers show potentiality of both male and female contraceptive [Bhargava, S. K. Fitoterapia 59, 163-177, 1988]. Flowers of this plant are also effective in leprosy, leucorrhoea and gout [The Wealth of India-Raw Materials pp. 341-346, 1988, PID, CSIR, New Delhi].

The seeds of the plant are used in Ayurvedic system as an anthelmintic drug [Kafti, M. C. T., Manjunath, B. L. J. Indian Chem. Soc. 6, 839-845, 1929; Raj, R. K., Kurup, P. A. Indian Journal of Medical Research 56, 1818-1825, 1968; Prashanth D; Asha M. K., Amit A., Padmaja, R. Fitoterpia 72, 421-422, 2001; Jain, J. P., Naqvi, S. M. A. J Res. Ayur Siddha 7, 13-22, 1986]. Significant anti-ovulatory and anti-implantation activities have also been reported in hot alcoholic extract of seeds when given to rats and rabbits. The active constituent has been identified as butin [Bhargava, S. K. Ethnopharmacology 18, 95-101, 1986]. Butin also exhibits male contraceptive properties [Dixit, V. P., Agrawal, M., Bhargava, S. K., Gupta, R. S., Jain, G. C. Lugoslavica Physiologica et Pharmacologica Acta 17, 151-162, 1981]. Antifertility effect of seed extract of Butea frondosa has also been reported in mice [Razdan, M. K., Kapila, K., Bhide, N. K. Indian Journal Physiology Pharmacology 14, 57-60, 1970, Razdan, M. K., Kapila, K., Bhide, N. K. Indian Journal Physiology Pharmacology 13, 239-249, 1969, Porwal, M.; Mehta, B. K., Gupta, D. N. National Academy Science Letters (India) 11, 81-84, 1988; Billore, K. V., Audichya, K. C. J. Res. Ind. Med. Yoga and Homeo. 13, 105, 1978]. Hemagglutinating activity is also reported in seeds of Butea frondosa showing specificity towards human erythrocytes [Bhalla, V., Walter, H. Research Bulletin of the Panjab University, Science 48, 87-94, 1999; Wongkham, S., Boonsiri, P., Trisonthi, C., Simasathiansophon, S., Wongkham, C., Atisook, K. Journal of Science Society of Thailand 21, 27-36, 1995]. The lectins such as Butea monosperma agglutinin (BMA) isolated from the seeds of Butea monosperma are responsible for agglutinating property [Ghosh, B., Dasgupta, B., Sircar, P. K. Indian Journal Biochemistry Biophysics 18, 166-169, 1981; Horejsi, V., Ticha, M., Novotny, J., Kocourek, J. Biochimica et Biophysica Acta 623, 439-448, 1980]. A petroleum ether extract of seeds of Butea frondosa showed growth regulating Duvenile hormone (JH)] activity against the fifth instar larvae of Dysdercus similis (F) [Kumar, B., Haresh, T. S. S. Journal Animal Morphology Physiology 36, 209-217, 1989]. The seed oil of B. monosperma shows significant bactericidal and fungicidal effect in in vitro testing [Mehta, B. K., Dubey, A., Bokadia, M. M., Mehta, S. C. Aata Microbiologica Hungarica 30, 75-77, 1983; Porwal, M., Sharma, S., Metha, B. K. Fitoterapia 59, 134-135, 1988]. Petroleum ether extract of Butea superba seeds exhibits anthelmintic and hypotensive activities [Siddiqui, H. H., Inamdar, M. C. Indian Journal of Pharmacy 25, 270-271, 1963]. Butea monosperma gum has also been found useful in cases of chronic diarrhea. It is a powerful astringent and also decreases bilirubin level [Rasheed, A., Alam. M. Tufail, M., Khan, F. Z. Hamdard Medicus 36, 36-39,1993].

### Pure Compounds

A variety of compounds have been isolated from Butea species. The root of Butea monosperma contains glucose, glycine, a glycoside (aglycon) and an aromatic hydroxy compound [Tandon, S. P., Tiwari, K. P., Saxena, and V. K. Proceedings of the National Academy of Sciences, India, Section A: Physical Sciences 39, 237-239, 1969]. From the tuber root of Butea superba, 3,7,3′-Trihydroxy-4′-methoxyflavone and 3,3′-dihydroxy-4′-methoxyflavone-7-O-β-D-glucopyranoside have been isolated [Roengsumran, S., Petsom, A., Ngamrojanavanich, N., Rugsilp, T., Sittiwicheanwong, P., Khorphueng, P., Cherdshewasart, W., Chaichantipyuth, C. Journal of Scientific Research of Chulalongkorn University 25,169-176, 2000].

From the stem of Butea monosperma, a flavonoid 8-C-prenylquercetin 7,4′-di-O-methyl-3-O-α-L-rhamnopyranosyl(1-4)-α-L-rhamnopyranoside has been isolated [Yadav, R. N., Singh, R. K. Journal of the Institution of Chemists (India) 70, 9-11,1998]. An anti-fungal compound isolated from the petroleum and ethyl acetate extract of the stem bark from Butea monosperma has been identified as (−)-3-hydroxy-9-methoxypterocarpan [(−)-medicarpin]. Both (−)-medicarpin and its acetate salt were active against Cladosporium cladosporioides. Its petroleum ether extract also yielded lupenone, lupeol and sitosterol. Two isoflavones isolated from the ethyl acetate extract were found to be 5-methoxygenistein and prunetin [Bandara, B. M. R., Kumar, N. S., Wimalasiri, K. M. S. Journal of the National Science Council of Sri Lanka 18, 97-103, 1990; Bandara B. M. R., Kumar N. S., Samaranayake K. M. Journal of Ethnopharmacology 25, 73-75, 1989]. In addition to stigmasterol-3-α-L-arabinopyranoside, four compounds isolated from the stem of Butea monosperma have been characterized as 3-methoxy-8,9-methylenedioxypterocarp-6-ene, 21-methylene-22-hydroxy-24-oxooctacosanoic acid Me ester, 4-pentacosanylphenol and pentacosanyl-β-D-glucopyranoside [Shukla, Y. N., Mishra, M., Kumar, S. Indian Journal of Chemistry, Section B 41B, 1283-1285, 2002], Stigmasterol, stigmasterol-β-D-glucopyranoside, nonacosanoic acid, 3α-hydroxyeuph-25-ene and 2,14-dihydroxy-11,12-dimethyl-8-oxo-octadec-11-enylcyclohexane were also isolated [Mishra, M., Shukla, Y. N., Kumar, S. Phytochemistry 54, 835-838, 2000]. The tetramers of leucocyanidin were isolated from the gum and the bark of Butea monosperma having —C—C— and —C—O—C— linkages [Seshadri, T. R., Trikha, R. K. Indian Journal of Chemistry 9, 1201-1203, 1971]. An antimicrobial flavonol glycoside, 3,5,7,3′,4′-pentahydroxy-8-methoxy-flavonol-3-O-α-D-xylopyranosyl (1-2)-α-L-rhamnopy ranoside [Yadava, R. N., Reddy, K. I. S. Journal of Asian Natural Products Research 1, 139-145, 1998] and 3,7-dihydroxy-8-methoxyflavone 7-O-α-L-rhamnopyranoside were isolated from the stem of Butea superba [Yadava, R. N.; Reddy, K. I. S. Fitoterapia 69, 269-270, 1998].

Two compounds, 3,9-dimethoxypterocarpan, and triterpenoid ester, 3α-hydroxyeuph-25-enyl heptacosanoate were isolated from the leaves of Butea monosperma [Shukla, Y. N., Mishra, M., Kumar, S. Indian Joumal of Chemistry, Section B 41B, 881-883, 2002].

Several flavonoids, butein, butin, butrin, isobutrin, isobutyine, coreopsin, isocoreopsin, sulfurein, monospermoside, isomonospermoside, palasitrin, 3′,4′,7-trihydroxyflavone [Mishra, M., Shukla, Y. N., Kumar, S. Journal of Medicinal and Aromatic Plant Sciences 24, 19-22, 2002; Gupta, S. R., Ravindranath, B., Seshadri, T. R. Phytochemistry 9, 2231-2235, 1970; Puri, B., Seshadri, T. R. Journal of Scientific & Industrial Research 12B, 462-466, 1953; Puri, B., Seshadri, T. R. Journal of Scientific & Industrial Research 14B, 1589-1592, 1955] were isolated from Butea monosperma flowers. Isobutrin and butrin, showed hepatoprotective activity [Wagner, H., Geyer, B., Fiebig, M., Kiso, Y., Hikino, H. Planta Medica 52, 77-79, 1986]. Butrin was also isolated from flowers of Butea superba [Rao, V. S., Seshadri, T. R. Journal of Scientific & Industrial Research 8B, 178-179, 1949]. Stigmasterol-3-β-D-glucopyranoside, γ-sitosterolglucoside, sitosterol [Mishra, M., Shukla, Y. N., Kumar, S. Journal of Medicinal and Aromatic Plant Sciences 24, 19-22, 2002; Murti, P., Bhaskara R., Seshadri, T. R. Proceedings—Indian Academy of Sciences, Section A 20A, 279-91, 1944; Murti, P., Bhaskara R., Seshadri, T. R. Proceedings—Indian Academy of Sciences, Section A (1941), 13A, 395-8] were reported from flowers of Butea monosperma. A triterpene (TBM) showing anticonvulsive activity [Kasture, V. S., Kasture, S. B., Chopde, C. T. Pharmacology, Biochemistry and Behavior 72, 965-972, 2002] and a tritepeneglycoside [Murti, P. B. R., Seshadri, T. R. Proceedings Indian Academy of Sciences, Section A 20A, 279-291, 1944] have also been isolated from the flowers of Butea monosperma. Myricyl alcohol, stearic, palmitic, arachidic and lignoceric acids [Murti, P. Bhaskara, R., Krishnaswamy, H. Proceedings—Indian Academy of Sciences, Section A 12A 472-476, 1940], glucose, fructose, histidine, aspartic acid, alanine and phenylalanine were also isolated from Butea frondosa flowers [Shah, K. C., Baxi, A. J., Dave, K. K. Indian Drugs 29, 422-3,1992].

From the seed extract of Butea monosperma, several flavonoids have been reported viz. 5,6,7,4′-tetrahydroxy-8-methoxyisoflavone 6-O-rhamnopyranoside [Saxena, V. K., Sharma, Devendra, N. Journal of the Institution of Chemists (India) 70, 218-220, 1998]. Butin isolated from seeds has also been reported to show both male and female antifertility activity [Bhargava, S. K. Journal of Ethnopharmacology 18, 95-101, 1986; Dixit, V. P., Agrawal, M., Bhargava, S. K., Gupta, R. S., Jain, G. C. lugoslavica Physiologica et Pharmacologica Acta 17, 151-162, 1981]. α-Amyrin, β-sitosterol, β-sitosterol-β-D-glucoside and sucrose were isolated from Butea frondosa seeds [Chandra, S., Lal, J., Sabir. M. Indian Joumal of Pharmacy 39, 79-80, 1977]. Palasonin, the anthelmintic principle was isolated from Butea frondosa seeds [Kumar, D., Mishra, S. K., Tandon, S. K., Tripathi, H. C. Indian Journal of Pharmacology 27, 161-166, 1995; Chandra, S., Lal, J., Sabir, M. Indian Journal of Pharmaceutical Sciences 40, 97-98, 1978; Raj, R. K., Kurup, P. A. Indian Journal of Medical Research 56, 1818-1825, 1968]. Monospermin [Mehta, B. K., Bokadia, M. M. Chemistry & Industry (London, U. K.) 98, 1981] and an acid imide [Barua, A. K., Chakrabarti, P. I., Das, K. G., Nair, M. S. B. Chemistry & Industry (London, U. K.) 1376, 1970] were isolated from seeds of Butea monosperma. An imide, palasonin-N-phenylimide was isolated from pods of Butea monosperma [Guha, P. K., Poi, R., Bhattacharyya, A. Phytochemistry 29, 2017, 1990]. 1-Carbomethoxy-2-carbomyl hydrazine [Sharma, S., Batra, A., Mehta, B. K. Indian Journal of Chemistry, Section B 30B, 15-16, 1991], 2-hydroxy-ω-methylallophanic acid [Porwal, M., Sharma, S., Mehta, B. K. Indian Journal of Chemistry, Section B 27B, 281-282, 1988], 4-carbomethoxy-3,6-dioxo-5-hydro-1,2,4-triazine [Porwal, M., Mehta, B. K., Gupta, D. N. National Academy Science Letters (India) 11, 81-84, 1988] were isolated from seed coats of Butea monosperma. Fatty acids such as myristic, palmitic, stearic, arachidic, behenic, lignoceric oleic, linoleic and linolenic were isolated from Butea monosperma seeds [Sengupta, A., Basu, S. P. Journal of the American Oil Chemists Society 55, 533-535, 1978]. 15-Hydroxypentacosanoic acid [Sharma, S., Batra, A., Mehta, B. K. Indian Journal of Chemistry, Section B 30B, 715-716, 1991], n-heneicosanoic acid δ-lactone [Bishnoi, P., Gupta, P. C. Planta Medica 35, 286-288, 1979] and 10,16-dihydroxyhexadecanoic acid [Chatterjea, J. N., Sengupta, S. C., Misra, G. S., Agarwal, S. C. Indian Journal of Chemistry, Section B 14B, 719-721, 1976] were isolated from seeds of Butea monosperma. Phosphatidylcholine, phosphatidylethanolamine and phosphatidylinositol were identified as major components in seeds of Butea monosperma [Prasad, R. B. N., Rao, Y. N., Rao, S. V. J. Am. Oil Chem. Soc. 64, 1424-1727, 1987]. Lectins isolated from the seeds of Butea monosperma exhibit agglutinating activity [Wongkham, S., Boonsiri, P., Trisonthi, C., Simasathiansophon, S., Wongkham, C., Atisook, K. Journal of the Science Society of Thailand 21, 27-36, 1995; Ghosh, B., Dasgupta, B., Sircar, P. K. Indian Journal of Biochemistry & Biophysics 18, 166-169, 1981]. The seeds oil of Butea parviflora has afforded the glycerides of palmitic, stearic, lignoceric, oleic and linoleic acids [Garg, S. K. Fette, Seifen, Anstrichmittel 73, 437-438,1971].

Four acid esters, jalaric ester I and II and laccijalaric ester I and II and aleuritic acid and aldehydic acids were isolated from the soft resin of Butea frondosa seedlac [Singh, A. N., Upadhye, A. B., Mhaskar, V. V., Dev, S. Tetrahedron 30, 867-874, 1974; Khurana, R. G.; Singh, A. N., Upadhye, A. B., Mhaskar, V. V., Dev, S. Tetrahedron 26, 4167-4175, 1970; Madhav, R., Seshadri, T. R., Subramanian, G. B. V. Indian Journal of Chemistry 5, 182-184, 1967].

A (+)-leucocyanidin, 3′,4′,5,7-tetrahydroxyflavan-3,4-diol and leucoantho-cyanidins [Ganguli, A. K., Seshadri, T. R. Tetrahedron 6, 21-23, 1959; Ganguli, A. K., Seshadri, T. R. Journal of Scientific & Industrial Research 17B, 168, 1958] and riboflavine and thiamine [Broker, R. I; Bhat, J. V. Current Science 22, 343, 1953] were isolated from Butea frondosa gum.

### Enforcement of Invention

Osteoporosis is one of the major problems in our aging society. Osteoporosis results in bone fracture in older members of the population, especially in post-menopausal women. In traditional medicine, there are many natural crude drugs that have the potential for use to treat bone diseases. So far, there is no report in literature on anti-osteoporosis (bone forming) activity of Butea species. It was thought to study the anti-osteoporotic activity of this plant.

From the foregoing discussion it would appear that there is an urgent need to discover and develop a drug of plant origin, which possess the ideal pharmacological profile and promote new bone formation. The Butea monosperma was a fit case to study such activity and the experiments show that it possesses promising bone forming activity.

Accordingly, the present invention provides new plant extracts, their fractions, subfractions, pure compounds isolated from these or other natural sources or synthesized, their pharmaceutically acceptable salts and compositions that are useful for the prevention or treatment of various medical indications associated with estrogen independent or dependent diseases or syndromes preferably in prevention or treatment of diseases and syndromes caused in humans and animals in particular:
a) Osteoporosis, bone loss, bone formation;
b) bone formation during Type-II/age related/senile osteoporosis, period of development and growth to attain higher peak bone mass, bone fracture healing, promotion of new bone formation in vitro/in vivo for replacement of defective bone;
d) estrogen related diseases or syndromes, preferably diseases or syndromes caused by an estrogen-deficient state in a mammal;
e) cardiovascular effects more particularly hyperlipidaemia, thrombosis and vasomotor system;
f) neurodegenerative effects such as stroke, senile dementia-Alzheimer type and Parkinson disease;
g) menopausal symptoms including hot flushes, urogenital atrophy, depression, mania, schizophrenia and the like, urinary incontinence, relief of dysmenorrhea; relief of dysfunctional uterine bleeding, an aid in ovarian development, treatment of acne and hirsutism;
h) cancers such as prostatic carcinoma, cancer of breast, cancer of uterus, cancer of the cervix and cancer of the colon;
i) control or regulation of fertility in humans and in other animals;
j) for use in the prevention of threatened or habitual abortion;
k) suppression of post-partum lactation;
l) physiological disorders such as obesity, depression etc.;

The present invention further relates to the processes for the preparation of pharmaceutically active extracts, fractions, subfractions, isolation of pure compounds, their pharmaceutically acceptable salts and compositions of the principal aspect of the present invention.

### SUMMARY OF INVENTION

Accordingly, the present invention provides a pharmaceutical composition for prevention or treatment of bone disorders comprising a therapeutically effective amount of the extract(s) or fraction(s) obtained from Butea species or compounds of formula 1 isolated therefrom or other natural sources or synthesized, their analogs or salts, either alone or in any combination in a ratio ranging from 1 to 10, optionally along with pharmaceutically acceptable excipient(s)

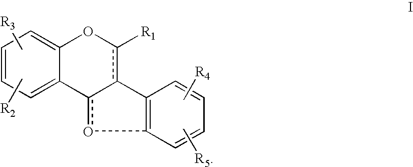

and the value of R₁, R₂, R₃, R₄ and R₅ in the compound of formula 1 being independently selected from the group consisting of hydrogen, methyl, hydroxy, methoxy group.
In an embodiment of the present invention, the compound(s) are selected from the group consisting of the compounds represented by the formulas K051, K052, K054, K080,K082,K095.

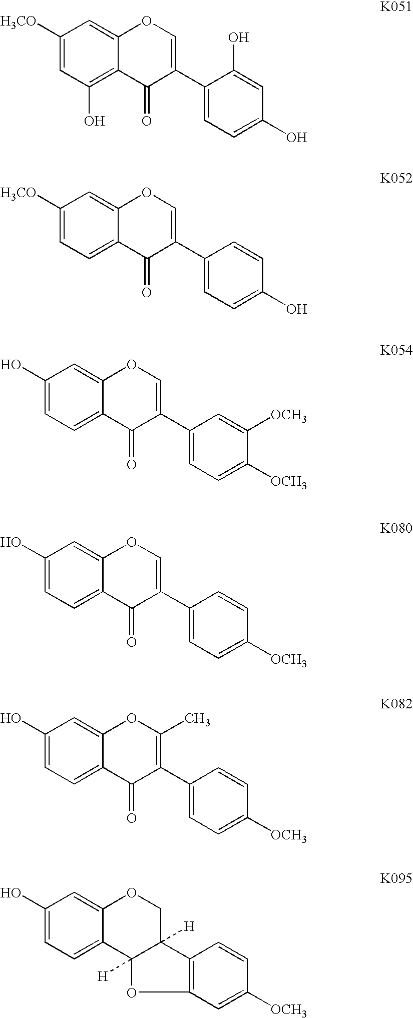

In another embodiment, the compounds are used either alone or in combination in the ratio ranging between 1 to 10 based on proportion, molar concentration or percent yield (FIGS. 11-15, Tables 10-11).
In another embodiment, the compounds K051 and K052 are used either alone or in combination based on molar concentration, percent yield, in equal or any proportions, preferably in equal proportions (FIGS. 11-15, Tables 10-11).
In another embodiment, the compounds K051, K052 and K095 are used either alone or in combination based on molar concentration, percent yield, in equal or any proportions, preferably in equal proportions (FIGS. 11-15, Tables 10-11).
In another embodiment, the compounds K054 and K080 are used either alone or in combination based on molar concentration, percent yield, in equal or any proportions, preferably in equal proportions (FIGS. 11-15, Tables 10-11).
In another embodiment, the compounds K051, K052, K054 and K080 are used either alone or in combination based on molar concentration, percent yield, in equal or any proportions, preferably in equal proportions (FIGS. 11-15, Tables 10-11).
In another embodiment, the compounds K051, K052, K054, K080 and K095 are used either alone or in combination based on molar concentration, percent yield, in equal or any proportions, preferably in equal proportions (FIGS. 11-15, Tables 10-11).
In another embodiment, the compounds K051, K052, K054, K080, K082 and K095 are used either alone or in combination based on molar concentration, percent yield, in equal or any proportions, preferably in equal proportions (FIGS. 11-15, Tables 10-11).
In another embodiment, the concentration of each compound used either alone or in combination is preferably 0.1 μM (FIGS. 11-15, Tables 10-11).
In another embodiment, the pharmaceutical diluent used is selected from the group consisting of lactose, mannitol, sorbitol, microcrystalline cellulose, sucrose, sodium citrate, dicalcium phosphate, or any other ingredient of the similar nature alone or in a suitable combination thereof.
In another embodiment, the pharmaceutical excipient used is selected from the group consisting of:
a) a diluent selected from lactose, mannitol, sorbitol, microcrystalline cellulose, sucrose, sodium citrate, dicalcium phosphate, or any other ingredient of the similar nature alone or in a suitable combination thereof;
b) a binder selected from gum tragacanth, gum acacia, methyl cellulose, gelatin, polyvinyl pyrrolidone, starch or any other ingredient of the similar nature alone or in a suitable combination thereof;
c) a disintegrating agent selected from agar-agar, calcium carbonate, sodium carbonate, silicates, alginic acid, corn starch, potato tapioca starch, primogel or any other ingredient of the similar nature alone or in a suitable combination thereof;
d) a lubricant selected from magnesium stearate, calcium stearate or steorotes, talc, solid polyethylene glycols, sodium lauryl sulphate or any other ingredient of the similar nature alone or in a suitable combination thereof;
e) a glidant selected from colloidal silicon dioxide or any other ingredient of the similar nature alone or in a suitable combination thereof;
f) a sweetening agent selected from sucrose, saccharin or any other ingredient of the similar nature alone or in a suitable combination thereof;
g) a flavoring agent selected from peppermint, methyl salicylate, orange flavor, vanilla flavor, or any other pharmaceutically acceptable flavor alone or in a suitable combination thereof;
h) a wetting agents selected from cetyl alcohol, glyceryl monostearate or any other pharmaceutically acceptable flavor alone or in a suitable combination thereof;
i) an absorbents selected from kaolin, bentonite clay or any other pharmaceutically acceptable flavor alone or in a suitable combination thereof; and
j) a solution retarding agents selected from wax, paraffin or any other pharmaceutically acceptable flavor alone or in a suitable combination thereof.
   In another embodiment, the effective dose of the composition is ranging between 0.1
   to 5000 mg per kg body weight preferably 1 mg to 500 mg per kg body weight, daily, bi-weekly, weekly or in more divided doses.
   In another embodiment, the composition is useful for the prevention or treatment of bone disorders such as any diseases and syndromes caused by osteoporosis, bone loss, bone formation, bone fracture healing, attainment of higher peak bone mass when administered during the period of growth, and promotion of new bone formation in vitro/in vivo.
   In another embodiment, the ethanolic extract of stem bark showed greater intensity in alkaline phosphatase staining when compared to corresponding vehicle (ethanol:DMSO, 50:50, v/v) control osteoblast cell cultures at time intervals of 24 h and 48 h (FIG. 1).
   In another embodiment, the ethanolic extract of stem bark exhibiting total alkaline phosphate activity higher by 58% as compared to 28% increase in enzyme activity in presence of sodium β-glycerophosphate per se treated bones (Table 1).
   In another embodiment, the ethanolic extract of stem bark induced marked proliferation of primary osteoblasts in culture when compared to corresponding vehicle control group at a concentration of 0.05% and 0.1% wherein the percent viable cells are 330% and 361%, respectively in comparison to that of vehicle control group taken as 100% (Table 2).
   In another embodiment, the ethanolic extract of stem bark at its osteogenic concentrations (0.05% and 0.1%), however, did not exhibit any proliferative effect on Ishikawa (human uterine glandular epithelial carcinoma) or MCF-7 (human cancer breast) cell lines (FIG. 2).
   In another embodiment, the osteoblast specific proliferation effect of the extract, demonstrates lack of any estrogen agonistic action of the extract at the endometrial and breast levels.
   In another embodiment, the ethanolic extract of stem bark exhibiting more than 2.5-fold increase in expression of collagen-I (a marker of osteoblast proliferation and differentiation) in calvaria of 21-day old rats 72 h after single 1000 mg/kg oral dose (FIG. 3).
   In another embodiment, the ethanolic extract of stem bark exhibiting more than 5 fold increase in the expression of osteocalcin, a marker of extracellular matrix maturation in the calvaria of 21-day old rats 72 h after single 1000 mg/kg oral dose (FIG. 4).
   In another embodiment, the ethanolic extract of stem bark exhibiting no effect of the treatment on expression of glyceraldehyde 3-phosphate dehydrogenase (GAPDH), a house-keeping gene (FIGS. 3 and 4).
   In another embodiment, the ethanolic extract of stem bark exhibiting increased rate of mineralization in vitro wherein higher intensity of alizarin red staining depicting increased deposition of nascent calcium in osteoblasts at both 24 h and 48 h with respect to corresponding vehicle controls (FIG. 5).
   In another embodiment, the ethanolic extract of stem bark exhibiting increased rate of mineralization in osteoblasts cultured for 7 days in vitro with respect to corresponding sodium β-glycerophosphate per se treated or vehicle control cultures, at a concentration of 0.1% (FIG. 6).
   In another embodiment, increased incidence of mineralised nodules in osteoblast cell cultures treated with ethanolic extract of stem bark for 15 and 25 days demonstrating increased rate of new bone formation (FIG. 7).
   In another embodiment, the higher intensity of alizarin staining was evident in long term osteoblast cell cultured on sterile bovine bone slices in the presence of ethanolic extract of stem bark for 18 and 30 days demonstrating increased rate of new bone formation (FIG. 8).
   In another embodiment, the ethanolic extract of stem bark on employing its effective osteogenic concentration, exhibited positive response by promoting bone formation as evidenced by T/C ratio of ≦0.5 in chick fetal bone culture assay (Table 3).
   In another embodiment, the ethanolic extract on employing or administering its effective osteogenic concentration did not inhibit PTH induced resorption of ⁴⁵Ca from chick fetal bones in culture with T/C ratio of 1.34, in comparison to T/C ratio of 0.66 and 0.37 in presence of 100 μM concentration of raloxifene and estradiol-17β (Table 4).
   In another embodiment, the ethanolic extract on oral administration at 1000 mg/kg daily dose for 30 consecutive days markedly increased (5% to 65%) bone mineral density (BMD) of all regions of Lumbar spine, femur and tibia bones of immature female Sprague-Dawley rats when compared with that of corresponding vehicle control group (Table 5).
   In another embodiment, the bones of immature rats treated with the extract also exhibiting higher mechanical strength as evidenced by greater force required to break the femur bone using three-pointing bending test for fracture and for compression of the Lumbar-3 vertebra using TK252C Muromachi Bone Strength Tester (Table 6).
   In another embodiment, the ethanolic extract on oral administration at 1000 mg/kg daily dose for 30 consecutive days markedly increased new bone formation as evidenced by double labeling technique involving administration of calcium seeking agents tetracycline at the time of start of treatment and calcein at the time of completion of treatment, sectioning of the undecalcified bones and visualisation of tetracycline label under UV light and calcein under orange filter (FIG. 9).
   In another embodiment, the ethanolic extract of stem bark is devoid of any estrogen agonistic activity at the uterine level when administered at 1000 mg/kg daily dose for 3 days in ovariectomized immature rats and 30 days in intact immature rats.
   In another embodiment, there was no effect of the ethanolic extract of stem bark on rate of age-related increase in body weight or uterine weight in immature rats (Tables 7 and 8).
   In another embodiment, the composition comprising ethanolic extract of seeds exhibited potent estrogen agonistic activity as evidenced by marked (433%) increase in uterine fresh weight in immature rat bioassay, comparable to that induced by 0.01 g/kg daily dose of ethynylestradiol (Table 8).
   In another embodiment, the ethanolic extract of stem bark at 1000 mg/kg daily dose administered for 3 consecutive days to ovariectomized immature rats produced 4% inhibition in 17α-ethynylestradiol (0.01 mg/kg/day) induced uterine weight gain, as compared to 37% inhibition observed with 0.25 mg/kg daily dose of the antiestrogen raloxifene (Table 9).
   In another embodiment, the Butea species is selected from the group consisting of Butea monosperma, Butea parviflora, Butea minor and Butea superba, preferably Butea monosperma.
   In another embodiment, the plant parts used from Butea monosperma is selected from stem bark, twigs, leaves, flowers, seeds, preferably stem bark.
   In another embodiment, the bioactive extract/fraction is selected from the group consisting of alcoholic extract, chloroform. soluble fraction, n-butanol soluble fraction (FIGS. 1-10; Tables 1-9).
   In another embodiment, n-butanol soluble and chloroform soluble fractions of the ethanolic extract of stem bark showed greater intensity in alkaline phosphatase staining when compared to corresponding vehicle (ethanol:DMSO, 50:50, v/v) control osteoblast cell cultures at 48 h (FIG. 10).
   In another embodiment, compounds K051, K052, K054, K080 and K095 increased expression of alkaline phosphatase (a marker of osteoblast differentiation), in osteoblasts plated on plastic cover slips (6 mm diameter) and incubated for 48 h in the concentration range of 10¹¹ M to 10⁻⁵ M when compared to corresponding vehicle control group (FIG. 11, Table 10).
   In another embodiment, compounds K051, K052, K054, K080 and K095 enhanced osteoblast cell proliferation after 24 h in concentration range of 10⁻¹¹ M to 10⁻⁵ M when compared to vehicle control group in MTT assay (FIG. 12, Table 11).
   In another embodiment, compounds K051, K052, K054, K080, K082 and K095 enhanced mineralisation as evidenced by increased deposition of nascent calcium in osteoblast cells cultured for 7 days and quantified by alizarin extraction method (FIG. 13).
   In another embodiment, the compounds K051, K052, K054, K080, K082 and K095 used either alone or in combination increased intensity of alizarin red staining, demonstrating increased rate of new bone formation, in osteoblasts cultured on sterile bovine bone slices in 96-well plate for 15 days (FIGS. 14 and 15).
   The invention further provides, a process for the preparation of bioactive fraction from Butea species as claimed in claim 1, wherein the process comprises:
a) soaking the powdered plant parts in alcoholic solvent and removing and concentrating the solvent by conventional methods to obtain alcoholic extract;
b) triturating the alcoholic extract obtained from step (a) with hexane to obtain the hexane soluble fraction and hexane insoluble fraction,
c) triturating the hexane insoluble fraction with chloroform to obtain chloroform soluble fraction and chloroform insoluble fraction,
d) subjecting the chloroform soluble fraction to repeated chromatography to obtain compounds K084, K090, K095, K103, K105, K113, K115,
e) partitioning the chloroform insoluble fraction with n-butanol and water to obtain n-butanol soluble fraction and aqueous fraction,
f) subjecting the n-butanol soluble fraction to repeated chromatography to obtain compounds K010, K039, K040, K051, K052, K053, K054, K064, K080, K082, K098, K111
   In another embodiment, the alcohol used for extraction is selected from the group consisting of methanol, ethanol, propanol or their appropriate mixtures thereof. In another embodiment, the chromatographic method used for isolation of compounds is selected from column, flash, medium pressure and HPLC.
   In another embodiment, the compounds may be converted to the pharmaceutically acceptable salts comprising of hydrochloride, formate, acetate, phenyl acetate, trifluroacetate, acrylate, ascorbate, benzoate, chlorobenzoates, bromobezoates, iodobenzoates, nitrobenzoates, hydroxybenzoates, alkylbenzoates, alkyloxybenzoates, alkoxycarbonylbenzoates, naphthalene-2 benzoate, butyrates, phenylbutyrates, hydroxybutyrates, caprate, caprylate, cinnamate, mandelate, mesylate, citrate, tartarate, fumerate, heptanoate, hippurate, lactate, malate, maleate, malonate, nicotinate, isonicotinate, oxalate, phthalate, terephthalate, phosphate, monohydrogen phosphate, dihydrogen phosphate, metaphosphate, pyrophosphate, propiolate, propionate, phenylpropionate, salicylate, sebacte, succinate, suberate, sulphate, bisulphate, pyrosulphate, sulphite, bisulphate, sulphonate, benzene sulphonate, bromobenzene sulphonates, chlorobenzene sulphonates, ethane sulphonates, methane sulphonates, naphthalene sulphonates, toluene sulphonates, and the likes.
   In another embodiment, the said method comprising the steps of administering to the subject in need a pharmaceutical composition as aforesaid, optionally along with pharmaceutically acceptable excipients.
   In another embodiment, the composition is administered by the route selected from oral, percutaneous, intramuscular, intraperitoneal, intravenous, local.
   In another embodiment, the composition is used in a dose ranging between 1 to 5000 mg/kg body weight.
   In another embodiment, the composition is used in the form of tablet, syrup, powder, capsule, suspension, solution, ointment, mixture.
   In accordance with the principal embodiment, the present invention provides new plant extracts, their fractions, subfractions, pure compounds isolated from these or other natural sources or synthesized, their pharmaceutically acceptable salts and compositions that are useful for the prevention or treatment of various medical indications associated with estrogen independent or dependent diseases or syndromes preferably in prevention or treatment of diseases and syndromes caused in humans and animals.
   In an important embodiment, the present invention provides a pharmaceutical composition comprising a therapeutically effective amount of new plant extracts, their fractions, subfractions, pure compounds isolated from these or other natural sources or synthesized, their pharmaceutically acceptable salts and compositions thereof, alone, in a mixture form or in a combination of a pharmacologically active or inactive agent or both and one or more pharmaceutically acceptable carrier or excipient.
   In another embodiment, the present invention provides a medical method of employing the new plant extracts, their fractions, subfractions, pure compounds isolated from these or other natural sources or synthesized, their pharmaceutically acceptable salts and pure compounds isolated from these or other natural sources or synthesized, their pharmaceutically acceptable salts and compositions thereof and methods of using such agents for the prevention or treatment of symptoms of estrogen dependent or independent states in mammals and animals, in particular osteoporosis, bone loss, bone formation and cardiovascular effects.
   In another embodiment of the medical methods of the present invention, the new plant extracts, their fractions, subfractions, pure compounds isolated from these or other natural sources or synthesized, their pharmaceutically acceptable salts and pure compounds isolated from these or other natural sources or synthesized, their pharmaceutically acceptable salts and compositions thereof are employed in the prevention or the treatment of estrogen dependent or estrogen independent cancers. In yet another alternative embodiment of the medical methods, the new plant extracts, their fractions, subfractions, pure compounds isolated from these or other natural sources or synthesized, their pharmaceutically acceptable salts and compositions of the present invention are employed in the prevention or the treatment of disease conditions or disorders associated with an aberrant physiological response to endogenous estrogen including control or regulation of fertility in humans and in other animals.

### BRIEF DESCRIPTION OF FIGURES

FIG. 1 Expression of alkaline phosphatase activity in osteoblasts cultured for 24 h and 48 h in presence of ethanolic extract of stem bark of Butea monosperma

FIG. 2 Proliferative activity of ethanolic extract of stem bark of Butea monosperma in Ishikawa (human uterine glandular epithelial carcinoma) and MCF-7 (human cancer breast) cell lines by MTT assay

FIG. 3 Transcript level of expression of collagen-I in calvaria of 21-day old rats 72 h after single 1000 mg/kg oral dose of ethanolic extract of stem bark of Butea monosperma

FIG. 4 Transcript level of expression of osteocalcin in calvaria of 21-day old rats 72 h after single 1000 mg/kg oral dose of ethanolic extract of stem bark of Butea monosperma

FIG. 5 Nascent calcium deposition in osteoblasts cultured for 24 and 48 h in presence of ethanolic extract of stem bark of Butea monosperma by Alizarin red staining

FIG. 6 Mineralisation in osteoblasts cultured for 7 days in presence of ethanolic extract of stem bark of Butea monosperma by von Kossa silver staining

FIG. 7 In vitro nodule formation by osteoblasts cultured for 15 and 25 days in presence of ethanolic extract of stem bark of Butea monosperma following von Kossa silver staining

FIG. 8 In vitro mineralisation and nodule formation by osteoblasts cultured on bovine bone slices for 18 and 30 days in presence of ethanolic extract of stem bark of Butea monosperma following Alizarin red staining

FIG. 9 Bone apposition rate in femur and tibia of immature rats treated with 1000 mg/kg dose of ethanolic extract of stem bark of Butea monosperma for 30 days using tetracycline and calcein labeling

FIG. 10 Alkaline phosphatase expression in osteoblasts cultured for 48 h in presence of different fractions of the ethanolic extract of stem bark of Butea monosperma

FIG. 11 Quantification of alkaline phosphatase activity in osteoblasts cultured in presence of varying concentration of pure compounds isolated from active ethanolic extract of stem bark of Butea monosperma

FIG. 12 Osteoblast cell proliferation cultured for 24 h in presence of varying concentration of pure compounds isolated from active ethanolic extract of stem bark of Butea monosperma using MTT assay

FIG. 13 Quantification of mineralization in osteoblasts cultured for 7 days in presence of varying concentration of pure compounds isolated from active ethanolic extract of stem bark of Butea monosperma by acetic acid extraction

FIG. 14 In vitro mineralisation by osteoblasts cultured on bovine bone slices for 15 days in presence of pure compounds isolated from active ethanolic extract of stem bark of Butea monosperma following Alizarin red staining

FIG. 15 In vitro mineralisation by osteoblasts cultured on bovine bone slices for 15 days in presence of pure compounds isolated from active ethanolic extract of stem bark of Butea monosperma mixed in equimolar concentration following Alizarin red staining

### DESCRIPTION OF THE INVENTION

The present invention provides new plant extracts, their fractions, subfractions, pure compounds isolated from these or other natural sources or synthesized, their pharmaceutically acceptable salts and pure compounds isolated from these or other natural sources or synthesized, their pharmaceutically acceptable salts and compositions and methods of using such agents for the prevention or treatment of symptoms of various medical indications associated with estrogen independent or dependent diseases or syndromes caused in humans and/or animals.

The term “pharmaceutically acceptable salts” as used throughout this specification and the appended claims denotes salts of the types disclosed in the article by Berge et al. (J. Phramaceutical Sciences, 66 (1), 1-19, 1977). Suitable pharmaceutically acceptable salts include salts formed by in-organic acids such as hydrochloric acid, hydrobromic acid, hydroiodic acid, nitric acid, sulphuric acid, phosphoric acid, hypophosphoric acid, and the like, as well as the salts derived from organic acids such as aliphatic mono and dicarboxylic acids, phenyl substituted alkanoic acids, aromatic acids, aliphatic and aromatic sulphonic acids. Such pharmaceutically acceptable acid addition salts include formate, acetate, phenyl acetate, trifluroacetate, acrylate, ascorbate, benzoate, chlorobenzoates, bromobezoates, iodobenzoates, nitrobenzoates, hydroxybenzoates, alkylbenzoates, alkyloxybenzoates, alkoxycarbonylbenzoates, naphthalene-2 benzoate, butyrates, phenylbutyrates, hydroxybutyrates, caprate, caprylate, cinnamate, mandelate, mesylate, citrate, tartarate, fumerate, heptanoate, hippurate, lactate, malate, maleate, malonate, nicotinate, isonicotinate, oxalate, phthalate, terephthalate, phosphate, monohydrogen phosphate, dihydrogen phosphate, metaphosphate, pyrophosphate, propiolate, propionate, phenylpropionate, salicylate, sebacte, succinate, suberate, sulphate, bisulphate, pyrosulphate, sulphite, bisulphate, sulphonate, benzene sulphonate, bromobenzene sulphonates, chlorobenzene sulphonates, ethane sulphonates, methane sulphonates, naphthalene sulphonates, toluene sulphonates, and the like. Most preferred salts are fumerate or ascorbate or hydrochloride.

The term “pharmaceutically acceptable compositions” of the agents of the present invention as used throughout this specification and the appended claims may be prepared by procedures known in the art using pharmaceutically acceptable excipients known in the art.

Methods of preventing or treating disorders or disease conditions mentioned herein comprise administering to an individual human being or any other mammal or any other animal in need of such treatment a therapeutically effective amount of one or more of the agents of this invention or a pharmaceutically acceptable salt or a pharmaceutically acceptable composition thereof with one or more of the pharmaceutically acceptable carriers, excipients etc.

The dosage regimen and the mode of administration of the agents of this invention or a pharmaceutically acceptable salt or a pharmaceutically acceptable composition thereof with one or more of the pharmaceutically acceptable carriers, excipients etc. will vary according to the type of disorder or disease conditions described herein and will be subject to the judgment of the medical practitioner involved.

The agent of this invention or a pharmaceutically acceptable salt or a pharmaceutically acceptable composition thereof with one or more of the pharmaceutically acceptable carriers, excipients etc. may be effectively administered in doses ranging from 0.1 mg to 5000 mg, more preferably in doses ranging from 0.5 to 1000 or still more preferably in the doses ranging from 1 mg to 500 mg weekly or bi-weekly or daily or twice a day or three times a day or in still more divided doses.

Therapeutically effective amounts of agents of the present invention or a pharmaceutically acceptable composition thereof may be enclosed in gelatin capsules or compressed into the tablets or pills or may be formulated in the form of lozenges, inclusion complexes with cyclodextrin derivatives, injectable depo formulations, aerosols, granules, powders, oral liquids, mucosal adhesive formulations, gel formulations, troches, elixirs, suspensions, syrups, wafers, liposomal delivery systems, implants, suppository, pessary, microemulsions, nanoemulsion, microparticles, nanoparticles, controlled release delivery systems, transdermal delivery systems, targeted delivery systems such as conjugates with monoclonal antibodies or with other suitable carrier moieties.

Such doses may be administered by any appropriate route for example, oral, systemic, local or topical delivery for example, intravenous, intra-arterial, intra-muscular, subcutaneous, intra-peritoneal, intra-dermal, buccal, intranasal, inhalation, vaginal, rectal, transdermal or any other suitable means in any conventional liquid or solid dosage form to achieve, conventional delivery, controlled delivery or targeted delivery of the compounds of this invention or a pharmaceutically acceptable salt or a pharmaceutically acceptable composition thereof with one or more of the pharmaceutically acceptable carriers, excipients etc.

A preferred mode of administration of agents of the present invention or a pharmaceutically acceptable salt or a pharmaceutically acceptable composition thereof is oral.

Oral compositions will generally comprise of agents of the present invention or a pharmaceutically acceptable composition thereof and one or more of the pharmaceutically acceptable excipients.

The oral compositions such as tablets, pills, capsules, powders, granules, and the like may contain any of the following pharmaceutically acceptable excipients:
1. a diluent such as lactose, mannitol, sorbitol, microcrystalline cellulose, sucrose, sodium citrate, dicalcium phosphate, or any other ingredient of the similar nature alone or in a suitable combination thereof;
2. a binder such as gum tragacanth, gum acacia, methyl cellulose, gelatin, polyvinyl pyrrolidone, starch or any other ingredient of the similar nature alone or in a suitable combination thereof;
3. a disintegrating agent such as agar-agar, calcium carbonate, sodium carbonate, silicates, alginic acid, corn starch, potato tapioca starch, primogel or any other ingredient of the similar nature alone or in a suitable combination thereof;
4. a lubricant such as magnesium stearate, calcium stearate or steorotes, talc, solid polyethylene glycols, sodium lauryl sulphate or any other ingredient of the similar nature alone or in a suitable combination thereof;
5. a glidant such as colloidal silicon dioxide or any other ingredient of the similar nature alone or in a suitable combination thereof;
6. a sweetening agent such as sucrose, saccharin or any other ingredient of the similar nature alone or in a suitable combination thereof;
7. a flavoring agent such as peppermint, methyl salicylate, orange flavor, vanilla flavor, or any other pharmaceutically acceptable flavor alone or in a suitable combination thereof;
8. wetting agents such as cetyl alcohol, glyceryl monostearate or any other pharmaceutically acceptable flavor alone or in a suitable combination thereof;
9. absorbents such as kaolin, bentonite clay or any other pharmaceutically acceptable flavor alone or in a suitable combination thereof;
10. solution retarding agents such as wax, paraffin or any other pharmaceutically acceptable flavor alone or in a suitable combination thereof.

### Test Procedure for Preparation of Extracts and Fractions

### Stem Bark of Butea Monosperma

Extraction with Ethanol

Powdered stem bark of Butea monosperma (5.5 kg) were placed in glass percolator with ethanol (25 L) and are allowed to stand at room temperature for about 16 hours (overnight). The percolate was collected. This process of extraction was repeated four times. The combined extract was filtered, concentrated at 45° C.; weight of extract obtained 380 g (6.90%, C003).

Partition of Ethanolic Extract

Ethanolic extract (300 g) was triturated with hexane (500 ml×15). The hexane soluble fraction was then concentrated under the reduced pressure at 40° C., weight of hexane fraction obtained 15.5 g (0.28%, F004). Residue obtained after triturating with hexane was then triturated with chloroform (400 ml×10). Chloroform soluble fraction was then concentrated under reduced pressure at 40° C., weight of chloroform fraction obtained 15 g (0.27%, F005). Residue obtained after successive extraction with hexane and chloroform was suspended in water (800 ml) in a separating funnel and extracted with n-butanol saturated with water (300 ml×14) and then concentrated under reduced pressure at 45° C. Weight of n-butanol fraction obtained 124.0 g (2.25%, F006). Water-soluble fraction concentrated under vacuum using rotavapor at 45° C., weight of aqueous fraction obtained 206.50 g (3.75%, F007).

Ethanolic extract (300 g) was triturated with hexane (500 ml×15). The hexane soluble fraction was then concentrated under the reduced pressure at 40° C., weight of hexane fraction obtained 15.5 g (0.28%, F004). Residue obtained after triturating with hexane was then triturated with chloroform (400 ml×10). Chloroform soluble fraction was then concentrated under reduced pressure at 40° C., weight of chloroform fraction obtained 15 g (0.27%, F005). Residue obtained after successive extraction with hexane and chloroform was suspended in water (800 ml) in a separating funnel and extracted with n-butanol saturated with water (300 ml×14) and then concentrated under reduced pressure at 45° C. Weight of n-butanol fraction obtained 124.0 g (2.25%, F006). Water-soluble fraction concentrated under vacuum using rotavapor at 45° C., weight of aqueous fraction obtained 206.50 g (3.75%, F007).

Twigs of Butea monosperma
Extraction with Ethanol

Powdered Butea monosperma twigs (1.0 kg) was placed in a glass percolator with ethanol (2.0 L) and was allowed to stand overnight at room temperature (about 16 hours). The percolate was separated and the process of extraction was repeated four times. The combined ethanolic extract was filtered and concentrated at 45° C. The concentrated extract, weighed, obtained 132.0 g (1.32%, A001).

Leaves of Butea monosperma
Extraction with Ethanol

Powdered Butea monosperma leaves (13.0 kg) was placed in a glass percolator with ethanol (20 L) and was allowed to stand overnight at room temperature (about 16 hours). The percolate was separated and the process of extraction was repeated four times. The combined ethanolic extract was filtered and concentrated at 45° C. The concentrated extract, weighed, obtained 1300 g (10%, C007)

Flowers of Butea monosperma
Extraction with Ethanol

Powdered Butea monosperma flowers (3.0 kg) was placed in a glass percolator with ethanol (15 L) and was allowed to stand overnight at room temperature (about 16 hours). The percolate was separated and the process of extraction was repeated four times. The combined ethanolic extract was filtered and concentrated at 45° C. The concentrated extract, weighed, obtained 430 g (14.33%).

Seeds of Butea monosperma
Extraction with Ethanol

Powdered Butea monosperma seeds (10.0 kg) was placed in a glass percolator with ethanol (14 L) and was allowed to stand overnight at room temperature (about 16 hours). The percolate was separated and the process of extraction was repeated four times. The combined ethanolic extract was filtered and concentrated at 45° C. The concentrated extract, weighed, obtained 1.75 kg (17.5%).

Test Procedure for Isolation of Compounds from Fractions of the Ethanolic Extract of Stem Bark of Butea monosperma

### Chloroform Soluble Fraction (F005)

Repeated column chromatography of chloroform soluble fraction (F005, 15.0 g) afforded seven compounds, K084, K090, K095, K103, K105, K113 and K115. These compounds were characterized from detailed spectroscopic studies. These compounds are known in the literature:

1. Physical and Spectral Data of K084 (2-methyl, 7-acetyloxy, 4′-methoxy isoflavones,)

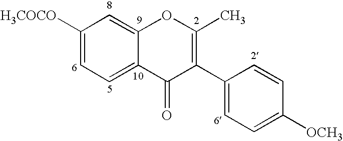

Yield: 64 mg. (0.00116%); mp: 155-156° C.; IR (KBR)νₘₐₓ: 3426, 1666, 1617, 1520, 1429, 1098, 1018 cm⁻¹; UV [symbol] ₘₐₓ nm: MeOH: 249, 266 and 340 nm; FAB-MS: m/z 325[M+H]⁺, 324[M]⁺, 283 [(M+H)—COCH₂]⁺, 282 [M—COCH₂]⁺, ¹H NMR: (CDCl₃, 200 MHz) δ: 8.23 (1H, d, J=8.6Hz, H-5), 7.15 (1H, dd, J=8.6, 1.7 Hz, H-6), 7.08 (1H, d, J=1.7 Hz, H-8), 7.20 (2H, d, J=8.5 Hz, H-2′), 6.97(2H, d, J=8.5 Hz, H-3′), 6.97(2H, d, J=8.5 Hz, H-5′), 7.20 (2H, d, J=8.5 Hz, H-6′), 2.31 (3H, s, 2-CH₃), 2.35 (3H, s, OCOCH₃), 3.84 (3H, s, 4′-OCH₃).

### 2. Physical and Spectral Data of K090 (Docosanoic Acid)

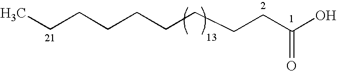

Yield: 2.0 g. (0.0363%); mp: 70-72° C.; IR (KBr) νₘₐₓ: 3430, 2919, 2850, 2362, 1693, 1594, 1468, 1351 cm⁻¹, EI-MS: m/z 340 [M]⁺, 325, 311, 297, 283, 269, 255, 241, 227, 213, 199, 185, 171, 157, 143, 129; ¹H NMR: (CDCl₃+DMSO-d₆, 200 MHz) δ 2.28 (2H, t, J=7.19 Hz, H₂-2), 1.60 (2H, m, H-3), 1.25 (36H, br s, H-4 to H-21), 0.87 (3H, t, J=6.2 Hz, H-22).
3. Physical and Spectral Data of K095 (3-hydroxy-9-methoxypterocarpans Commonly Known as Medicarpin)

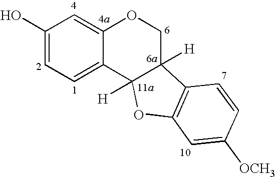

Yield: 100 mg. (0.00181%); mp: 127-128° C.; [α]²² _{D}: −226° (c, 0.1, CHCl₃); IR (KBr)νₘₐₓ: 3404, 2949, 1619, 1600, 1499, 1474, 1365, 1281, 1149, 1028, 934, 837, 764 cm⁻¹; UV λₘₐₓ nm: (MeOH) 282, 287; FAB-MS: C₁₆H₁₄O₄, m/z 270 [M]⁺; ¹H NMR: (CDCl₃, 200 MHz) δ: 7.37 (1H, d, J=8.3 Hz, H-1), 6.54 (1H, d, J=8.3 Hz, H-2), 6.44-6.41 (1H, m, H-4), 3.67-3.51 (1H, m, H-6), 4.23 (1H, dd, J=6.0, 9.8 Hz, H-6), 3.67-3.51(1H, m, H-6a), 7.12 (1H, d, J=8.7 Hz, H-7), 6.56-6.41 (1H, m, H-8), 6.56-6.41 (1H, m, H-10), 5.49 (1H, d, J=6.0 Hz, H-11a), 3.76 (3H, s, 9-OCH₃). pos 4. Physical and Spectral Data of K103 (3-methoxy-8,9 methylenedioxy coumestan Commonly Known as Flemmichapparin C)

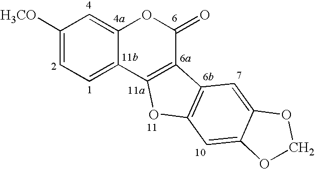

Yield: 5 mg. (0.00009%); mp: 272° C.; IR (KBr) νₘₐₓ: 1740, 1608, 1501, 1473, 1359, 1274, 1235, 1147, 1036, 942 cm⁻¹; UV [symbol] ₘₐₓnm: (MeOH) 340,310, 296 and 245; EI-MS: C₁₇H₁₀O₆, m/z 310 [M]⁺, 295 [M-CH₃]⁺, 267 [M-CH₃—CO]⁺; ¹H NMR: (DMSO-d₆, 200 MHz) δ: 7.85 (1H, d, J=8.0 Hz. H-1), 6.97-6.92 (2H, brd, J=9.1 Hz, H-2), 6.97-6.92 (2H, brd, J=9.1Hz, H-4), 7.57 (1H, s, H-7), 7.29 (1H, s, H-10), 6.17 (2H, s, O—CH₂—O), 3.88 (3H, s, 3-OCH₃).
5. Physical and Spectral Data of K105 (3-methoxy-8, 9-methylenedioxy-6a, 11a-dehydropterocarpan)

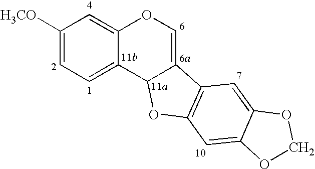

Yield: 42 mg. (0.0.00076%); mp: 163-164° C., [ [symbol] ]²⁸_{D}: −220° (c, 0.1, CHCl₃); IR (KBr) vₘₐₓ: 1664, 1614, 1566, 1496, 1459, 1380, 1226, 1134, 1029, 944,841,785 cm⁻¹; UV [symbol] ₘₐₓnm: (MeOH) 340, 288, 259; FAB-MS: C₁₇H₁₂O₅: m/z 296 [M]⁺, 281 [M-CH₃]⁺, and 265 [M-OCH₃]⁺; ¹H NMR: (CDCl₃, 200 MHz) δ: 7.36 (1H, d, J=8.0 Hz, H-1), 6.54-6.50 (2H, br d, J=8.5 Hz, H-2), 6.54-6.50 (2H, br d, J=8.5 Hz, H-4), 7.01 (1H, s, H-6), 7.26 (1H, s, H-7), 6.72 (1H, s, H-10), 5.51 (1H, s, H-11a), 5.99 (2H, s, O—CH₂—O), 3.84 (3H, s, 3-OCH₃).
6. Physical and Spectral Data of K113 (lupeonone (lup-20(29)-en-3-one)

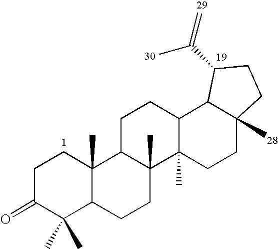

Yield: 38 mg. (0.00069%); mp: 168-170° C.; [ [symbol] ]³⁰_{D}: +60.6° (c, 0.5, CHCl₃); IR (KBr)νₘₐₓ: 1704 and 1630 cm⁻¹; EI-MS: C₃₀H₄₈O: m/z 424 [M]⁺; ¹H NMR: (CDCl₃, 200 MHz) [symbol] [symbol] 4.68 (1H, s, H-29 [symbol] ), 4.57 (1H, s, H-29 [symbol] ), 2.41 (1H, m, H-2), 2.38 (1H, m, H-19), 1.89 (1H, m, H-21β) 1.68 (3H, s, H-30), 1.07 (3H, s, H-26), 1.02 (3H, s, H-26), 0.99 (3H, s, H-27), 0.95 (3H, s, H-25), 0.93 (3H, s, H-28), 0.79 (3H, s, H-24).
7. Physical and Spectral Data of K115 (lupeol (lup-20(29)-en-3 [symbol] -ol)

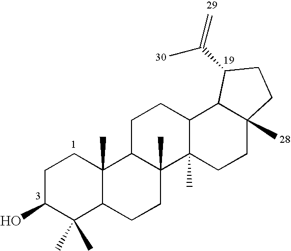

Yield: 40 mg. (0.00072%); mp: 210-212° C.; [α]³⁰_{D}: +27° (c, 1.2, CHCl₃); IR (KBr)νₘₐₓ: 3400, 2939, 1639, 1458, 1382 and 1035 cm⁻¹; FAB-MS: C₃₀H₅₀O: m/z 427 [M+H]⁺, 411, 409, 385, 221, 219, 207, 189, 136; ¹H NMR: (CDCl₃, 200 MHz) [symbol] 4.68 (1H, s, H-29 [symbol] ), 4.56 (1H, s, H-29 [symbol] ), 3.17 (1H, m, H-3), 2.39(1H, m, H-19), 1.9 (1H, m, H-21 [symbol] ), 1.67 (3H, s, H-30), 1.03 (3H, s, H-26), 0.96 (3H, s, H-26), 0.94 (3H, s, H-27), 0.82 (3H, s, H-25), 0.78 (3H, s, H-28), 0.76 (3H, s, H-24).
Test Procedure for Isolation of Compounds from n-butanol Soluble Fraction (F005) of the Ethanolic Extract of Stem Bark of Butea monosperma

Repeated column chromatography of n-butanol soluble fraction (100.0 g) afforded twelve compounds, K010, K039, K040, K051, K052, K053, K054, K064, K080, K082, K098 and K111. These compounds were characterized from detailed spectroscopic studies. These compounds are known in the literature:

8. Physical and Spectral Data of K010 (pentacosanoic acid 2,3-dihydroxy-propyl ester)

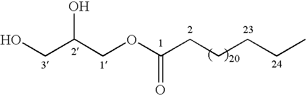

Yield: 175 mg. (00.00072%); mp: 72-74° C.; [ [symbol] ]²²_{D}: −3.11 (methanol+CHCl₃, c, 0.22); IR (KBr)νₘₐₓ: 3225, 1733, 1704, 1389, 725 cm⁻¹; FAB-MS: m/z 457 [M+H]⁺; ¹H NMR: (CDCl₃+DMSO-d₆, 200 MHz) δ 4.11 (2H, t, J=6.2 Hz, H-1′), 3.80 (1H, m, H-2′), 3.34 (2H, m, H-3′), 2.26 (2H, t, J=7.3 Hz, H-2), 1.56 (4H, H-23, 24), 1.25 (40H, br s, H-3 to H-22) 0.87 (3H, t, J=6.0 Hz, CH₃).
9. Physical and Spectral Data of K039 (2′- hydroxy genistein)

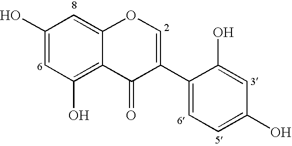

Yield: 25 mg. (0.00045%); mp: 270-273° C.; IR (KBr)νₘₐₓ: 3350, 1655, 1575, 1500, 1464,1234, 1178, 1104 cm⁻¹; UV [symbol] ₘₐₓ nm: MeOH: 315(sh), 258; MeOH+AlCl₃: 315(sh), 268; MeOH+AlCl₃-HCl: 315(sh), 268; FAB-MS: C₁₅H₁₀O₆: m/z 287 [M+H]⁺; ¹H NMR: (DMSO-d₆, 300 MHz) δ: 8.13 (1H, s, H-2), 6.36 (1H, d, J=1.5 Hz, H-6), 6.20 (1H, d, J=1.5 Hz, H-8), 6.34(1H, d, J=2.1Hz, H-3′), 6.25 (1H, d, J=8.4, 2.1 Hz, H-5′), 6.95 (1H, d, J=8.4 Hz, H-6′).
10. Physical and Spectral Data of K040 (7,4′-dihydroxy isoflavone Commonly Known as Daidzein)

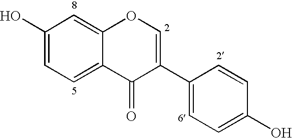

Yield: 70 mg.(0.0012%); mp: 330° C.; IR (KBr)νₘₐₓ: 3230, 2362, 1631, 1596, 1517, 1461, 1385, 1352, 1279, 1242, 1191,1096 cm⁻¹; UV [symbol] ₘₐₓ nm: MeOH: 303 (sh), 259 (sh), 249, 238(sh); FAB-MS: C₁₅H₁₀O₄: m/z 255 [M+H]⁺; ¹H NMR: (DMSO-d₆, 200 MHz) δ: 8.35 (1H, s, H-2), 8.04 (1H, dd, J=8.7 Hz, H-5), 7.01 (1H, d, J=8.7,1.6 Hz, H-6), 6.92 (1H, d, J=1.6 Hz, H-8), 7.44 (2H, d, J=8.4 Hz, H-2′), 6.86 (2H, d, J=8.4Hz, H-3′), 6.86 (2H, d, J=8.4 Hz, H-5′), 7.44 (2H, d, J=8.4 Hz, H-6′), 9.62 (1H, br hump, 7-OH).
11. Physical and Spectral Data of K051 (2′,4′,5-trihydroxy-7-methoxy isoflavones, commonly known as cajanin)

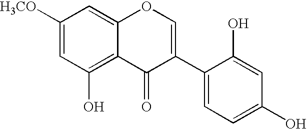

Yield: 20 mg. (0.00036%); mp: 215-216° C.; IR (KBr)νₘₐₓ: 3428, 1638, 1571, 1465 cm⁻¹; UV [symbol] ₘₐₓ nm: MeOH: 256, 218; FAB-MS : C₁₆H₁₂O₆: m/z 301 [M+H]⁺; ¹H NMR: (DMSO-d₆, 200 MHz) δ: 8.21 (1H, s, H-2), 6.62 (1H, d, J=1.6 Hz, H-6), 6.39 (1H, d, J=1.6 Hz, H-8), 6.35 (1H, br d, J=1.6 Hz, H-3′), 6.26 (1H, dd, J=8.2, 1.6 Hz, H-5′), 6.97 (1H, d, J=8.2 Hz, H-6′), 12.98 (1H, s, 5-OH), 3.84(3H, s, 7-OCH₃).
12. Physical and Spectral Data of K052 (4′-hydroxy, 7-methoxy-isoflavone commonly known as isoformonentin)

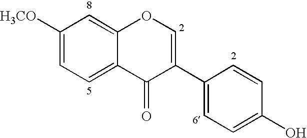

Yield: 65 mg. (0.00110%); mp: 218-220° C.; IR (KBr)νₘₐₓ: 3251, 2362, 1723, 1624, 1586, 1515, 1441, 1379, 1172, 1098, 1018 cm⁻¹; UV [symbol] ₘₐₛ nm: (MeOH) 317(sh), 256; (MeOH+NaOAc) 318(sh), 258; (MeOH+NaOMe) 343 (sh), 267; FAB-MS: C₁₆H₁₂O₄: m/z 269 [M+H]⁺; ¹H NMR: (DMSO-d₆, 200 MHz) δ: 8.40 (1H, s, H-2), 8.05 (1H, d, J=8.8 Hz, H-5), 7.13 (2H, m, H-6), 7.13 (2H, m, H-8), 7.43 (2H, d, J=8.3 Hz, H-2′), 6.84 (2H, d, J=8.3 Hz, H-3′), 6.84 (2H, d, J=8.3 Hz, H-5′), 7.43 (2H, d, J=8.3 Hz, H-6′), 3.93 (3H, s, 7-OCH₃), 9.57 (1H, s, 4′-OH).
13. Physical and Spectral Data of K053 (4′,5, 7-trihydroxy isoflavone, Commonly Known as Genistein)

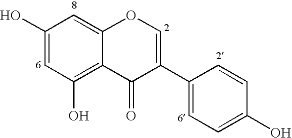

Yield: 25 mg. (0.00045%); mp: 301-302° C.; IR (KBr)νₘₐₓ: 3430, 2920, 1650, 1617, 1571, 1510, 1465, 1240, 1188, 1170 cm⁻¹; UV [symbol] ₘₐₓ nm: MeOH: 337, 262; FAB-MS: C₁₅H₁₀O₅: m/z 271 [M+H]⁺; ¹H NMR: (DMSO-d₆, 200 MHz) δ: 8.31 (1H, s, H-2), [symbol] [symbol] [symbol] (1H, d, J=1.8 Hz, H-6 ), 6.21 (1H, d, J=1.8 Hz, H-8 ), 7.72 (2H, d, J=8.4 Hz, H-2′), 6.81 (2H,d, J=8.4 Hz, H-3′), 6.81 (2H,d, J=8.4 Hz, H-5′), 7.72 (2H,d, J=8.4 Hz, H-6′), 12.94 (1H, s, 5-OH), 9.56 (1H, br hump, 7-OH).
14. Physical and Spectral Data of K054 (7, 3′-dihydroxy-4′-methoxyisoflavone, Commonly Known as Calycosin)

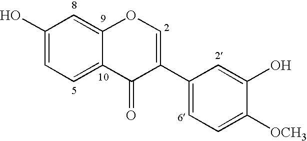

Yield: 15 mg. (0.00027%); mp: 245-247° C.; IR (KBr)νₘₐₓ: 3420, 1624, 1580, 1510, 1470, 1381, 1023, 853 cm⁻¹; UV [symbol] ₘₐₓ nm: MeOH: 288, 247, 224; MeOH +NaOAc: 327, 255, 221; NaOAc+boric acid: 288, 247, 225; FAB-MS: C₁₆H₁₂O₅: m/z 285 [M+H]⁺; ¹H NMR: (DMSO-d₆, 200 MHz) δ: 8.33 (1H, s, H-2), 8.03 (1H, d, J=8.7 Hz, H-5), 7.02-6.97 (1H, m, H-6), 6.92 (1H, d, J=2.0 Hz, H-8), 7.09 (1H, s, H-2′), 7.02-6.97 (2H, m, H-5′, 6′), 3.84 (3H, s, 4′-OCH₃), 9.10 (1H, s, 7-OH).
15. Physical and Spectral Data of K064 (nonacosanoic acid 2′,3′-dihydroxy-propyl ester)

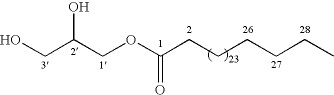

Yield: 150 mg. (0.00270%); mp: 90-91° C.; [ [symbol] ]²²_{D}: −3.87 (methanol+CHCl₃, c, 0.10); IR (KBr)νₘₐₓ: 3425, 2919, 2851, 2363, 1734, 1634, 1468, 1179, 1051, 720 cm⁻¹; FAB-MS: C₃₂H₆₄O₄: m/z 512[M]⁺, ¹H NMR: (CDCl₃+DMSO-d₆, 200 MHz) δ 4.04 (2H, t, J=5.3 Hz, H-1′), 3.80 (1H, m, H-2′), 3.48 (2H, m, H-3′), 2.29 (2H, t, J=7.3 Hz, H-2), 1.56 (6H, br m, H-26, 27, 28), 1.24 (46H, br s, H-3 to H-25) 0.87 (3H, t, J=6.0 Hz, CH₃).
16. Physical and Spectral Data of K080 (7-hydroxy, 4′-methoxy-isoflavone Commonly Known as Formonentin)

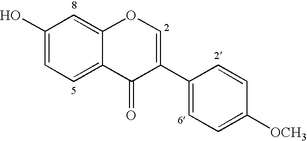

Yield: 105 mg. (0.00190%); mp: 258° C.; IR (KBr)νₘₐₓ: 3424, 2339, 1626, 1600, 1513, 1453, 1384, 1314, 1250, 1181, 1025 cm⁻¹; UV [symbol] ₘₐₓ nm: MeOH: 305, 250; MeOH+NaOAc: 262, 310, 340; FAB-MS: C₁₆H₁₂O₄: m/z 269 [M+H]⁺; ¹H NMR: (Acetone-d₆, 200 MHz) δ: 8.17 (1H, s, H-2), 8.07 (1H,d, J=8.7 Hz, H-5), 6.99 (1H, dd, J=9.2, 2.1 Hz, H-6), 6.90 (1H, d, J=2.0 Hz, H-8), 7.56.(2H, d, J=8.7 Hz, H-2′), 6.97 (2H, d, J=8.7 Hz, H-3′), 6.97(2H, d, J=8.7 Hz, H-5′), 7.56 (2H, d, J=8.7 Hz, H-6′), 3.83 (3H, s, 4′-OCH₃), 9.72 (1H, s, 7-OH).
17. Physical and Spectral Data of K082 (2-methyl, 7-hydroxy, 4′-methoxy isoflavone)

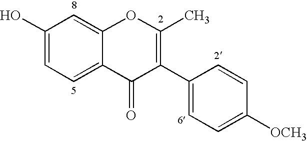

Yield: 08 mg. (0.00014%); mp: 240-242° C.; IR (KBr)νₘₐₓ: 3431, 1730, 1631, 1628, 1417, 1090, 1007 cm⁻¹; UV [symbol] ₘₐₓ nm: MeOH: 351, 269, 254; FAB-MS: C₁₇H₁₄O₄: m/z 283 [M+H]⁺; ¹H NMR: (DMSO-d₆, 200 MHz) δ: 7.89 [symbol] d, J=8.6 Hz, H-5), 6.91 [symbol] dd, J=8.6, 2.0 Hz, H-6), 6.84 (1H, d, J=2.0 Hz, H-8), 7.21 (2H, d, J=8.6 Hz, H-2′), 6.99 (2H, d, J=8.6 Hz, H-3′), 6.99 (2H, d, J=8.6 Hz, H-5′), 7.21 (2H, d, J=8.5 Hz, H-6′), 2.25. [symbol] [symbol] s, CH₃-2), 3.813 [symbol] s, 4′-OCH₃).
18. Physical and Spectral Data of K098 (4′,5-dihydroxy, 7-methoxy isoflavone Commonly Known as Prunetin)

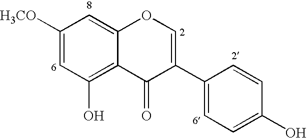

Yield: 15 mg. (0.00175%); mp: 240° C.; IR (KBr)νₘₐₓ: 3389, 2919, 1640, 1617, 1594, 1468, 1384, 1352, 1259, 1181, 1050 cm⁻¹; UV [symbol] ₘₐₓ nm: MeOH: 211, 258; MeOH+NaOAc: 212, 258; MeOH+NaOMe: 210, 270; El-MS: C₁₆H₁₂O₅: m/z 284 [M]⁺; ¹H NMR: (DMSO-d₆, 200 MHz) δ: 8.37 (1H, s, H-2), 6.62 (1H, d, J=2.2 Hz, H-6), 6.39 (1H, d, J=2.2 Hz, H-8), 7.38 (2H, d, J=8.5 Hz, H-2′), 6.82 (2H, d, J=8.3 Hz, H-3′), 6.82 (2H, d, J=8.3 Hz, H-5′), 7.38 (2H, d, J=8.5 Hz, H-6′), 3.85 (3H, s, 7-OCH₃), 12.94 (1H, s, 5-OH), 9.66 (1H, s, 4′-OH).
19. Physical and Spectral Data of K111 (formonentin 7-O-β-D-glycopyranoside Commonly Known as Ononin)

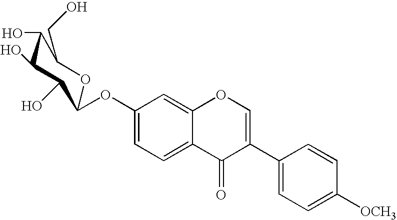

Yield: 35 mg. (0.00063%); mp: 218-219° C.; [ [symbol] ]²⁵_{D}: −24.2° (c, 0.11, pyridine) C₁₅H₁₀O₅; IR (KBr)νₘₐₓ: 3416, 1724, 1597, 1510, 1361, 1072, 774 cm⁻¹; UV [symbol] ₘₐₓ nm: MeOH 250 sh, 258, 302 sh; MeOH+NaOAc 259, 305 sh; MeOH+NaOMe 251 sh, 259, 302 sh; EI-MS: C₂₂H₂₂O₉: m/z 430 [M]⁺, 268 [M−sugar]⁺; ES-MS: m/z 453 [M+Na]⁺, 883 [2M+Na]⁺, 269 [M−sugar+H]⁺; ¹H NMR: (DMSO-d₆, 200 MHz) δ: 8.21 (1H, s, H-2), 7.84 (1H, d, J=8.4 Hz, H-5), 7.02 (1H, dd, J=8.4, 1.5 Hz. H-6), 6.95 (1H, d, J=1.5 Hz, H-8), 7.31 (2H, d, J=8.5 Hz, H-2′), 6.75(2H, d, J=8.5 Hz, H-3′), 6.75(2H, d, J=8.5 Hz, H-5′), 7.31 (2H, d, J=8.5 Hz, H-6′), 5.08 (1H, d, J=6.1 Hz, H-1″), 5.04-3.36 (6H, m, H-2″,3″,4″,5″,6″), 3.57 (3H, s, 4′-OCH₃).

### Biological Evaluation

The plant extracts/fractions/sub-fractions/pure compounds of the present invention were evaluated for use for enhancement of osteogenesis or bone formation, prevention or treatment of symptoms of estrogen deficiency or deprivation including estrogen deficient or deprivation state in mammals, in particular osteoporosis, bone formation, bone loss in humans and in other animals. Detailed procedures for the evaluation of the ethanolic extract of stem bark and its fractions and isolated compounds of the present invention are described hereunder. In preliminary evaluation, ethanolic extracts of twigs, leaves, flowers and seeds were either found to be inactive or showed low order of activity and were, therefore, not pursued. In addition, ethanolic extract of seeds exhibited potent estrogen agonistic activity. The activity testing illustrated in the following examples should, however, not be construed to limit the scope of invention.

### Test Procedure for the Determination of Osteogenic or Bone Forming Activity

Test solutions of the test extracts of the present invention are prepared in appropriate solvents in concentration range of 0.001% to 1%, most preferably in concentration of 0.1% of the present invention are prepared in appropriate solvents. 3-5 μl of each concentration are used for evaluation of bone forming in vitro. In control experiments, equal quantity of appropriate solvent is used in lieu of the test agent.

### Osteoblast Cell Culture

Osteoblasts arise from pluripotent mesenchymal stem cells. During culture, osteoblasts undergo three main phases with the expression of stage specific genes. These are:

Proliferation & differentiation Alkaline phosphatase, Collagen-I, Osterix, cbfa1: Days 1-12

Extra-cellular matrix maturation Osteocalcin, Osteopontin, Fibronectin: Days 12-18

Mineralization Calcification (nodule formation): Days 14-35

Neonatal mouse calvarial cell cultures are prepared as described previously (Endocrinology 139:4743) using slight modification. Briefly, for primary osteoblast cell cultures, frontal and parietal bones from Balb/c mouse neonates (1-3 day old) are digested in 0.1% collagenase/0.1% dispase in α-MEM to obtain 5 sequential digests. The second through fifth digests are combined and grown to confluence at 37° C. and 5% CO₂ in air in α-MEM, supplemented with 10% fetal bovine serum (FBS), 2 mM glutamine, 100 U/ml penicillin-streptomycin, Non-essential amino acid solution and sodium pyruvate. The effect of test agents is analysed using the following tests:

### a) Osteoblast Proliferation and Differentiation

### Expression of Alkaline Phosphatase Activity

### In Cultured Osteoblasts

Cells (˜10⁴ cells) plated on plastic cover slips (6 mm diameter) are incubated in the presence or absence of test agent for 24 h and 48 h, fixed in formalin and alkaline phosphatase (ALP) activity is displayed by incubation with ALP substrate solution (5 mg naphthol AS-MX phosphate, 0.25 ml ethylene glycol monomethyl ether, 10 mg Fast red TR, in 24 ml of 0.1 M TBS, pH 9.5) for 1 h at room temperature.

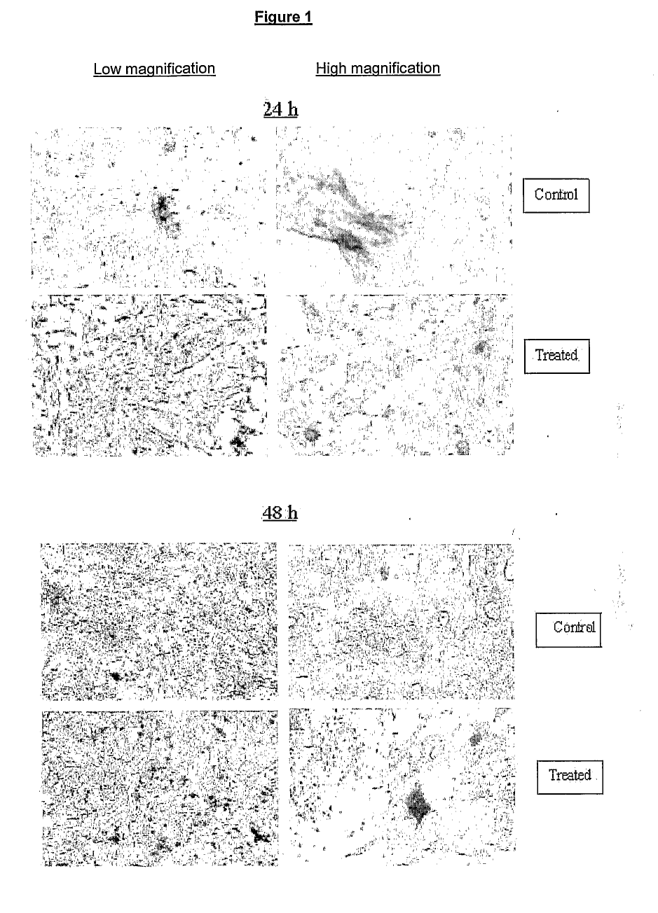

Cells cultured in presence of ethanolic extract of stem bark of Butea monosperma showed greater intensity in alkaline phosphatase staining when compared to corresponding vehicle (ethanol:DMSO, 50:50, v/v) control cultures at both the time intervals (FIG. 1).

### In Cultured Rat Fetal Bones

Long bones of 19 day old rat foetuses are isolated and cultured in BGJ_{b} medium in the presence of 100 μM glycerophosphate and/or extract of present invention for 48 h and then homogenized in 9 volumes of 50 mM Tris (pH 7.5) containing 0.1% Triton X-100. The homogenate is centrifuged at 5000 rpm for 10 min at 4° C. The supernatant is used as the enzyme solution. The activity of alkaline phosphatase is measured using ALP kit (Roche, Germany) and p-nitrophenol phosphate as substrate.

Total alkaline phosphate activity was found to higher by 58% in presence of ethanolic extract of stem bark of Butea monosperma as compared only 28% increase in enzyme activity in presence of sodium p-glycerophosphate per se treated bones Table 1).

TABLE 1
Total alkaline phosphatase activity in rat fetal
bones cultured for 48 h in presence of ethanolic
extract of stem bark of Butea monosperma

| | Total alkaline |
|---|---|
| | phosphatase activity* |
| Sodium β-glycerophosphate | 28% |
| (100 mM) | |
| Sodium β-glycerophosphate + | 58% |
| ethanolic extract of stem bark | |
| of Butea monosperma (0.1%) | |

*Percent increase (percent of vehicle control group)

### MTT Assay for Cell Proliferation

### Primary Osteoblast Cell Culture

MTT assay is a common assay used for assessing cell proliferation where tetrazolium salt is reduced to formazone crystals by the mitochondrial dehydrogenase enzyme, which are then dissolved in DMSO. More the number of metabolically active cells more will be the formazone crystals formed. Briefly, osteoblast cells are maintained in α-MEM medium supplemented with 10% FCS and 1% antibiotic solution in 96 well plate. When the cells attain 40% confluency, they are treated in presence or absence of test agents in 2% FCS supplemented media for 24 h. Twenty-four hours thereafter, MTT salt is added to the cells. After 4 h, the formazone crystals formed are dissolved in DMSO and readings taken at wavelength of 570 nm.

The extract at 0.05 (330%) and 0.1% (361%) concentrations induced marked proliferation of primary osteoblasts in culture when compared to corresponding vehicle control group (Table 2).

TABLE 2
Proliferative activity of ethanolic extract of stem
bark of Butea monosperma in primary osteoblasts isolated
from neonatal rat calvaria by MTT assay

| Concentration | | |
|---|---|---|
| of the extract (%) | Optical Density | % Viable cells |
| Vehicle | 0.415 ± 0.038 | 100 |
| 0.001 | 0.514 ± 0.028 | 123 |
| 0.005 | 0.541 ± 0.023 | 130 |
| 0.01 | 0.553 ± 0.031 | 133 |
| 0.05 | 1.373 ± 0.170 | 330 |
| 0.1 | 1.500 ± 0.197 | 361 |
| DMSO (0.1%) | 0.398 ± 0.064 | 95 |

### Ishikawa and MCF-7 Cell Lines

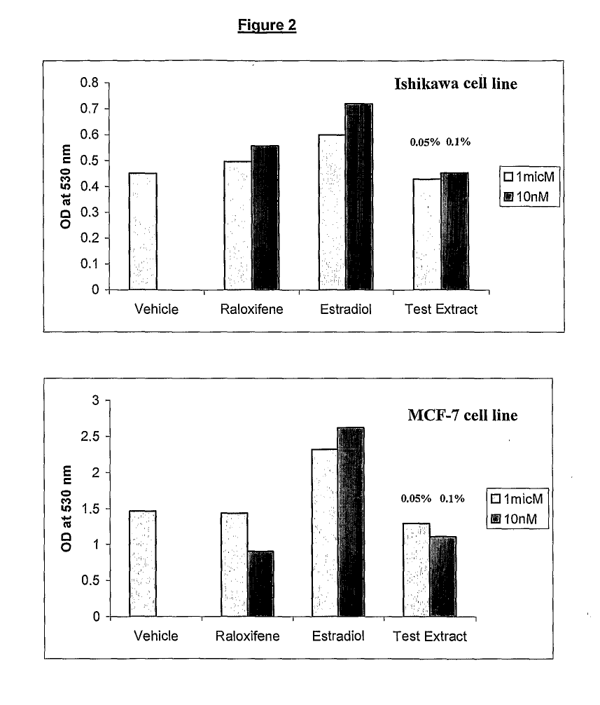

The extract at its osteogenic concentrations (0.05% and 0.1%), however, did not exhibit any proliferative effect on Ishikawa (human uterine glandular epithelial carcinoma) or MCF-7 (human cancer breast) cell lines. In comparison, while estradiol-17β (10 nM and 1 μM) induced marked increase in proliferation of both MCF-7 and Ishikawa cells, in case of raloxifene (10 nM and 1 μM), increased proliferation was observed in only the Ishikawa cell line, confirming its reported estrogenic effect at the uterine/endometrial level (FIG. 2). These findings while suggesting osteoblast specific proliferation effect of the extract, demonstrates lack of any estrogen agonistic action of the extract at the endometrial and breast levels and should, therefore, be devoid of ERT/HRT related health hazards.

### Expression of Collagen-I

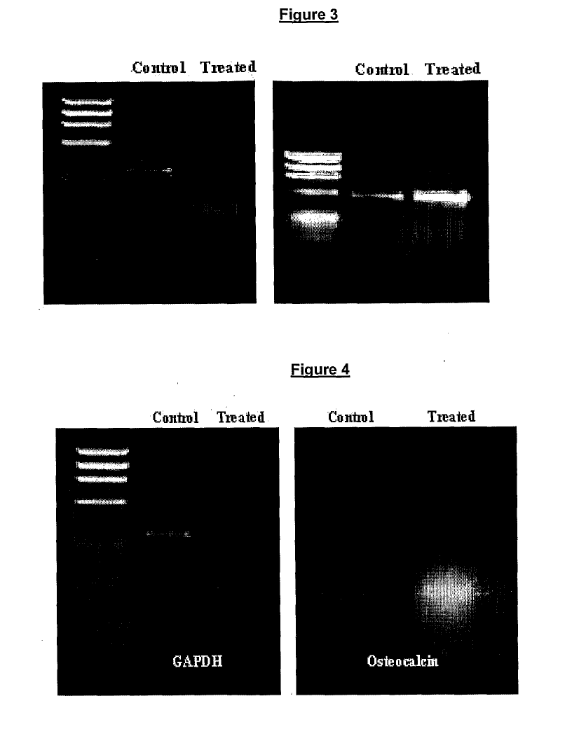

More than 2.5-fold increase in expression of collagen-I (a marker of osteoblast proliferation and differentiation) was also evident in calvaria of 21-day old rats 72 h after single 1000 mg/kg oral dose of ethanolic extract of stem bark of Butea monosperma. There was no effect of the treatment on GAPDH, a house-keeping gene (FIG. 3)

### b). Extra-Cellular Matrix Maturation Phase

Expression of Osteocalcin

This was associated with more than 5 fold increase in the expression of osteocalcin, a marker of extracellular matrix maturation in the calvaria of 21-day old rats 72 h after single 1000 mg/kg oral dose of ethanolic extract of stem bark of Butea monosperma (FIG. 4).

### c). Mineralisation

### Alizarin Red Staining of Osteoblasts

Cells are seeded onto plastic cover slips (6 mm, Thermanox, Nunc, USA) in 96- well plate and treated with culture medium containing α-MEM, supplemented with 10% fetal bovine serum, 2 mM glutamine, 100 U/ml penicillin-streptomycin, non-essential amino acid solution, sodium pyruvate, 10 mM p-glycerophosphate and 50 μg/ml ascorbic acid. After 24 and 48 h, the cell cultures are washed twice with cold PBS, fixed in cold 70% ethanol for 1 h, washed once with water and stained with 40 mM Alizarin red (pH 4.7) for 30 min and then washed in PBS to remove excess staining.

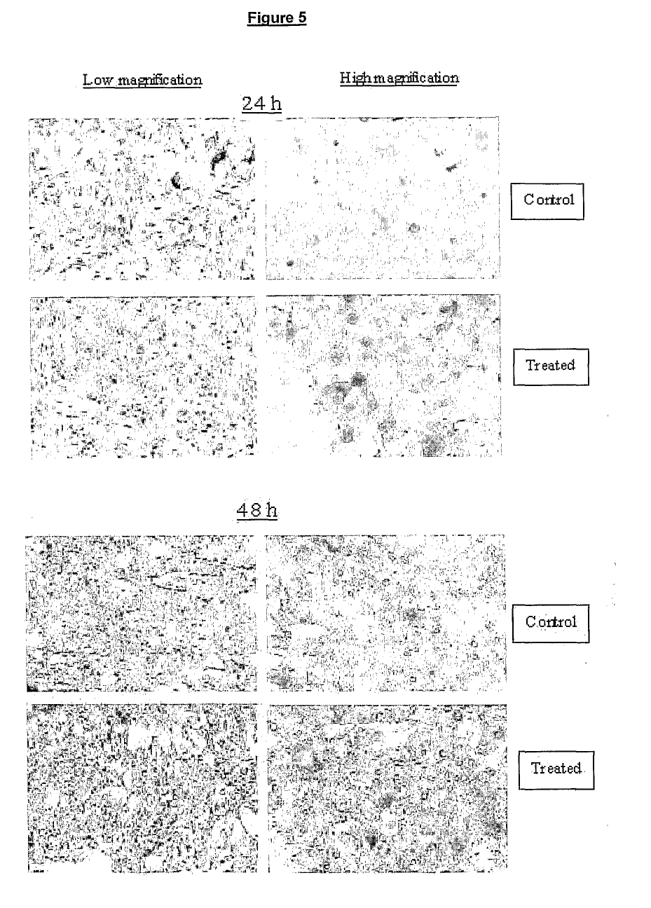

Cultures treated with the ethanolic extract of stem bark of Butea monosperma showed higher intensity of alizarin red staining depicting increased deposition of nascent calcium in osteoblasts at both 24 and 48 h with respect to corresponding vehicle controls, signifying increased rate of mineralization in vitro in presence of ethanolic extract of stem bark of Butea monosperma (FIG. 5).

### von Kossa Silver Staining: In Vitro Calcium Deposition Detection

### von Kossa Silver Staining of Osteoblasts

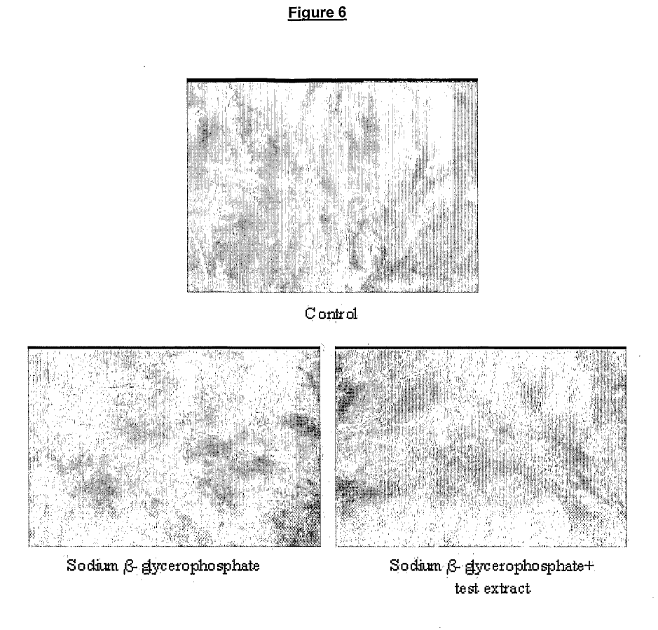

Cells seeded onto plastic cover slips were cultured for 7 days in the presence or absence of ethanolic extract of stem bark of Butea monosperma at a final concentration of 0.1% and stained with von Kossa silver staining. Cultures treated with the ethanolic extract of stem bark of Butea monosperma showed higher intensity of staining as well as cell proliferation with respect to corresponding sodium β-glycerophosphate per se treated or vehicle control cultures, signifying increased rate of mineralization in vitro in presence of ethanolic extract of stem bark of Butea monosperma (FIG. 6).

### Bone Nodule Formation Assay

### von Kossa Silver Staining

Cells are seeded onto plastic cover slips (6 mm, Thermanox, Nunc, USA) in 96-well plate and treated with the culture medium containing α-MEM, supplemented with 10% fetal bovine serum, 2 mM glutamine, 100 U/ml penicillin-streptomycin, non-essential amino acid solution, sodium pyruvate, 10 mM p-glycerophosphate and 50 μg/ml ascorbic acid. Culture medium is changed every alternate day. At termination of the culture, cells are washed twice with PBS, fixed in phosphate buffered formalin for 10 min, washed once with water and serially dehydrated in 70%, 95% and 100% ethanol (two times each) and air dried. The plates are then rehydrated in 100% to 95% to 80% ethanol to water. Silver nitrate (2% solution) is added and the plate is exposed to sunlight for 30 min after which the plate is rinsed with water. Sodium thiosulfate (5%) is added and after 3 min, the plates are rinsed in water.

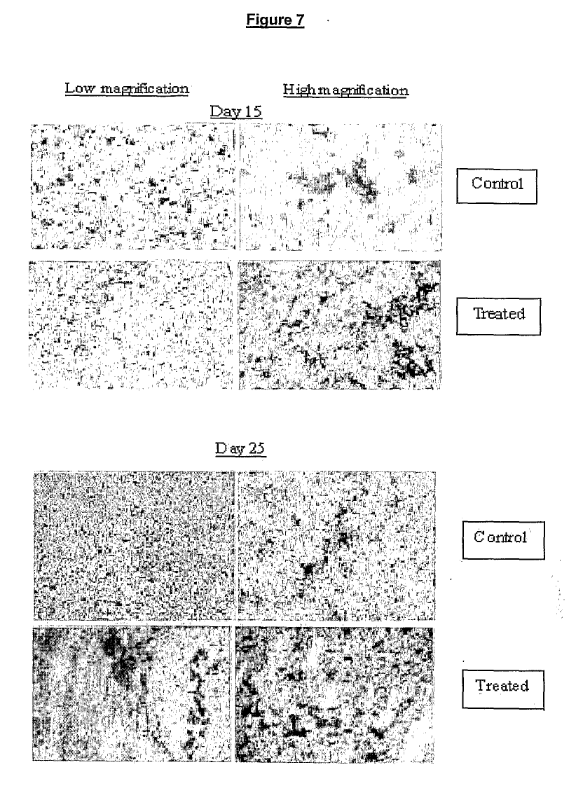

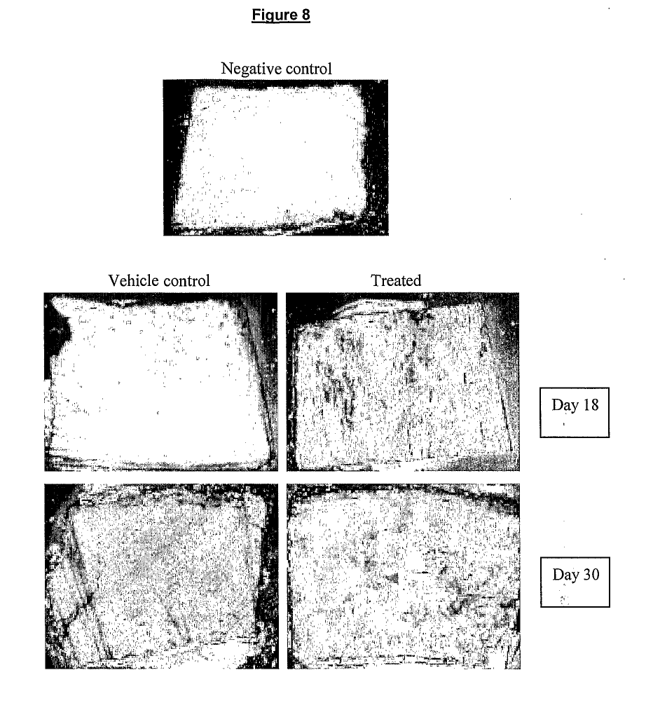

There was an increased incidence of mineralised nodules in cultures treated with the ethanolic extract of stem bark of Butea monosperma for 15 and 25 days demonstrating increased rate of new bone formation (FIG. 7).

### Alizarin Red Staining

Cells are seeded onto sterile bovine bone slices in 96-well plate and treated with the culture medium containing α-MEM, supplemented with 10% fetal bovine serum, 2 mM glutamine, 100 U/ml penicillin-streptomycin, non-essential amino acid solution, sodium pyruvate, 10 mM P-glycerophosphate and 50 μg/ml ascorbic acid. Culture medium is changed every alternate day. At termination of the culture, cells are washed twice with PBS, fixed in cold 70% ethanol for 1 h and washed once with water and stained with 40 mM Alizarin red (pH 4.7) for 30 min and then washed in PBS to remove excess staining. Higher intensity of colour was evident in cultures in presence of the extract demonstrating increased rate of new bone formation (FIG. 8).

### Chick Fetal Bone Culture Assay

A slight modification of the method of Raisz (Nature 197: 1015-1016, 1963) was employed. Femur bones are isolated from chick embryos on day 11 post-ovulation and cleared of adhering connective tissue by carefully rotating each bone on dry Whatman (I) filter paper under stereomicroscope. Each femur bone is then placed in a drop of PBS before culturing in BGJb medium (pH 7.3) supplemented with penicillin (0.075 mg/ml), streptomycin (0.05 mg/ml), HEPES (2.382 mg/ml) and BSA (1 mg/ml), transferred to BGJb culture medium containing ⁴⁵CaCl₂ (0.5 μCi/300 μl culture medium) and incubated for 2 h at 37° C. under 5% CO₂ in air. Labeled femur bones are then washed with BGJb medium for 24 h at 37° C. under 5% CO₂ in air. An aliquot of the medium was withdrawn for the measurement of ⁴⁵Ca released into the medium from bone during the first 24 h. Labeled bones are then transferred to BGJb medium containing parathyroid hormone (0.4 μM) and chase cultured for 96 h in the presence or absence of the test agent or the vehicle in 1 ml of BGJ_{b} medium. Appropriate solvents are selected from solvents like water, normal saline, phosphate buffered saline, phosphate buffer, ethanol, DMSO (final concentration 0.1%) alone or in a suitable combination thereof.

Contralateral femur of each fetus serves as corresponding control. Culture medium with the respective treatment in each well is changed after 48 h. An aliquot of the medium was withdrawn for the measurement of ⁴⁵Ca released into the medium from bone at 24, 48 and 96 h of treatment. On termination of the culture at 96 h, bones are transferred to 0.1 N HCl for 24 h. Radioactivity due to ⁴⁵Ca in the spent medium collected at 24, 48 and 96 h of culture and HCl extract at 96 h of culture is quantified by Liquid Scintillation Spectrophotometer in 10 ml of the scintillation fluid (ACS II scintillation cocktail, Amersham Biosciences, UK). This test procedure was used to determine bone forming as well as antiresorbing activity of the test agents.

### Bone Forming Activity

Bone forming activity was expressed by the following formula:

Bone forming activity (cpm)=A×(A/B)^{1/2} ×Total radioactivity incorporated into bone during 24 h incubation with ⁴⁵Ca

where total radioactivity refers to ⁴⁵Ca released into the medium during first 24 hrs incubation+⁴⁵Ca released into the medium during 24 to 96 hr of incubation+⁴⁵Ca remaining in the femur. A and B are the percent of ⁴⁵Ca remaining in the bone at 24 and 96 h, respectively of culture.

In accordance with the above test procedure, the extract on employing its effective osteogenic concentration, exhibits positive response by promoting bone formation as evidenced by T/C ratio of ≦0.5 (Table 3). T/C ratio close to unity indicates lack of any bone forming activity. Parathyroid hormone (PTH; aa 1-34), an osteogenic agent, was taken as positive control. Activity in the above test procedure indicates that the extract of the present invention is useful as bone forming agent in the treatment of osteoporosis caused by decreased rate of bone formation and for fracture healing.

TABLE 3
Bone forming activity of ethanolic extract of stem bark of
Butea monosperma using labeled chick fetal bones in culture

| Test agent | Concentration | T/C ratio |
|---|---|---|
| PTH (aa 1-34) | 0.4 μM | 0.44 |
| Ethanolic extract of stem bark | 0.1% | 0.34 |

### Bone Antiresorbing Activity

Bone anti-resorbing activity is expressed as percentage of ⁴⁵Ca released into the culture medium and the effect of the agent of invention as percent of the corresponding contra-lateral control or T/C ratio as shown below. Raloxifene and estradiol-17β, the known antiresorbing agents, were used as positive control. T/C ratio close to unity indicates lack of any antiresorbing activity.

$$T \text{/} C ratio = \frac{{}^{45} Ca resorption in presence of PTH + test agent }{{}^{45} Ca resorption in presence of PTH + vehicle}$$

In accordance with the above test procedure, the extract of the present invention on employing or administering its effective osteogenic concentration did not inhibit PTH induced resorption of ⁴⁵Ca from chick fetal bones in culture with T/C ratio of 1.34, in comparison to T/C ratio of 0.66 and 0.37 in presence of 100 μM concentration of raloxifene and estradiol-17β (Table 4).

TABLE 4
Evaluation of PTH-induced resorption of ⁴⁵Ca
from chick fetal bones in culture by ethanolic
extract of stem bark of Butea monosperma

| Test agent | Concentration | T/C ratio |
|---|---|---|
| Vehicle | — | 1.03 |
| Ethanolic extract of stem bark | 0.1% | 1.34 |
| Raloxifene | 100 μM | 0.66 |
| Estradiol-17β | 100 μM | 0.37 |

### Osteogenic Activity in Vivo

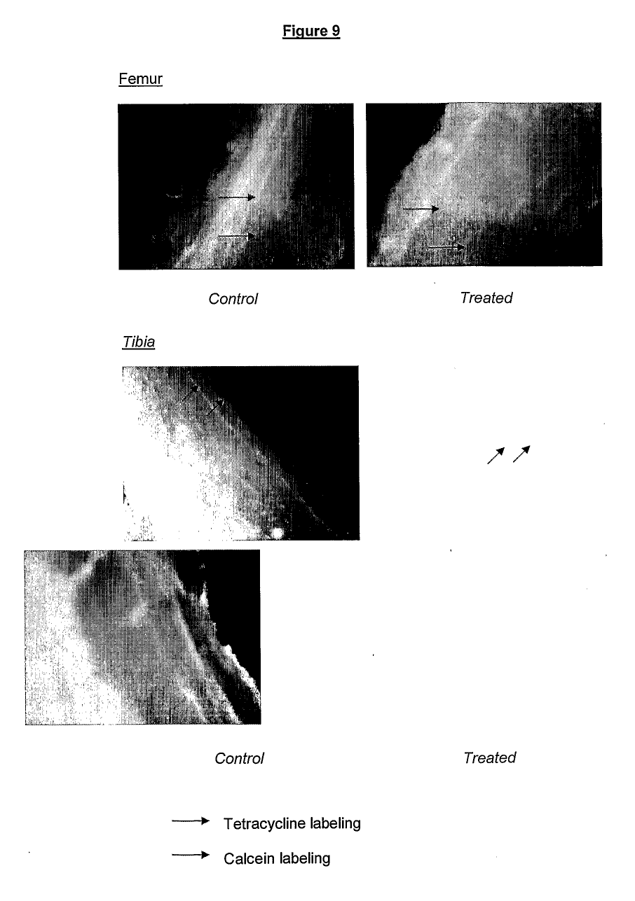

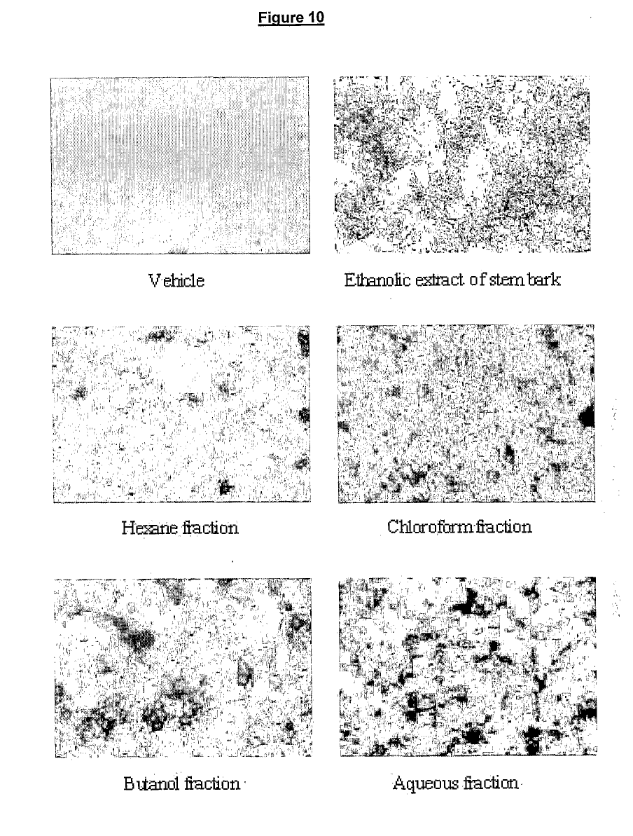

Twenty-one day old immature female Sprague-Dawley rats were randomized and treated with 1000 mg/kg daily dose of the extract or the vehicle (gum acacia in distilled water) by oral route for 30 consecutive days. For assessment of bone formation was done by Bone Mineral Density (BMD) measurement, mechanical strength and histomorphometry. The animals are autopsied on day 31 and lumbar vertebrae, femur and tibia bones were isolated, cleaned, fixed in 70% ethanol in saline and stored at −20° C. until BMD measurement. BMD measurements were performed using identical regions of interest (lumbar: global, L₁-L₄; femur: global, neck and mid-shaft; tibia: global, proximal and region about 2 mm proximal to tibio-fibular separation point) on an Hologic QDR-4500A fan-beam densitometer calibrated daily with Hologic hydroxyapatite anthropomorphic spine phantom using manufacturer provided software for small animals and scan speed of 1 mm/sec (4 lines/mm; Table 5). The mechanical properties of femur bone using three-pointing bending test for fracture and for compression of the Lumbar-3 vertebra of these rats were tested using TK252C Muromachi Bone strength tester (Table 6). For histomorphometry, each rat was administered tetracycline at the start of treatment and calcein at the time of completion of treatment, sectioning of the undecalcified bones and visualisation of tetracycline label under UV light and calcein under orange filter. Tetracycline and calcein are calcium-seeking agents (FIG. 9). Initial and final body weight and uterine weight of each rat were also recorded at autopsy (Table 7).

### Bone Mineral Density Measurement

Oral administration of the extract at 1000 mg/kg daily dose for 30 consecutive days markedly increased BMD of all regions of Lumbar spine, femur and tibia bones of immature female Sprague-Dawley rats when compared with that of corresponding vehicle control group (Table 5).

### Mechanical Properties

The bones of immature rats treated with the extract also showed higher mechanical strength as evidenced by greater force required to break the femur bone using three-pointing bending test for fracture and for compression of the Lumbar-3 vertebra using TK252C Muromachi Bone strength tester (Table 6).

TABLE 6
Mechanical properties of isolated bones of rats treated with 1000
mg/kg/day dose of ethanolic extract of stem bark of Butea monosperma
for 30 days using TK252C Muromachi Bone strength tester

| | Bending force | | Compression | |
|---|---|---|---|---|
| | Right Femur bone | | Lumbar-3 vertebra | |
| Treatment | Ultimate | Stiffness | Ultimate | Stiffness |
| group | force (N) | (N/mm) | force (N) | (N/mm) |
| Vehicle | 23.5 ± 1.0 | 29.8 ± 1.4 | 249.0 ± 76.7 | 326.0 ± 61.3 |
| Ethanolic extract | 26.8 ± 0.6 | 44.0 ± 5.7 | 395.3 ± 5.5 | 576.3 ± 26.3 |
| of stem bark of | | | | |
| Butea monosperma | | | | |

Crosshead speed for all the tests was 5 mm/min
Ultimate force is the maximum force at breaking point using three-point bending test for fracture

Crosshead Speed for all the Tests was 5 mm/min Ultimate force is the maximum force at breaking point using three-point bending test for fracture

### Tetracycline and Calcein Labeling

Femur and tibia bones of immature rats treated with the extract (1000 mg/kg for 30 days, po) also showed increased rate of bone formation as evidenced by double labeling technique involving administration of calcium seeking agents tetracycline at the time of start of treatment and calcein at the time of completion of treatment, sectioning of the undecalcified bones and visualisation of tetracycline label under UV light and calcein under orange filter (FIG. 9).

### Body Weight and Uterine Weight

There was no effect of the extract on rate of age-related increase in body weight or uterine weight (Table 7). Findings suggest lack of any estrogen agonistic activity of the extract at the tested dose and schedule in rats.

TABLE 7
Table 7. Change in age related body weight and uterine
weight in female Sprague-Dawley rats treated with ethanolic
extract of stem bark of Butea monosperma or the vehicle
for 30 consecutive days beginning day 21 of age

| | | | Uterine weight (mg) | |
|---|---|---|---|---|
| | Body weight (g) | | | /100 g |
| Treatment | Initial | Final | Absolute | body weight |
| Vehicle | 26.2 ± 1.5 | 63.3 ± 3.3 | 33.0 ± 0.002 | 52.4 ± 0.003 |
| Ethanolic extract | 27.1 ± 1.8 | 61.7 ± 4.2 | 32.3 ± 0.002 | 53.1 ± 0.004 |
| of stem bark | | | | |
| 1000 mg/kg/day, po | | | | |

### Hormonal Properties

### Estrogen Agonistic Activity

Estrogen agonistic activity of ethanolic extract of stem bark, leaves and seed of Butea monosperma and fractions was evaluated in bilaterally ovariectomized immature rats. The test agents were administered once daily for 3 consecutive days by the oral route and uterine weight gain over the corresponding vehicle control group was determined. 17α-Ethynylestradiol was used as reference standard. Ethanolic extract of stem bark (7%), twigs (23%) and leaves (44%) of this plant exhibited negligible to weak uterotrophic effect. In comparison, ethanolic extract of the seeds of this plant induced marked (433%) increase in uterine fresh weight and the effect was almost comparable to that induced by 0.01 g/kg daily dose of 17α-ethynylestradiol. Negligible to weak uterine weight gain was also observed in n-butanol soluble fraction of the ethanolic extract of stem bark (Table 8).

TABLE 8
Estrogen agonistic activity evaluation of extracts/fractions
of aerial parts of Butea monosperma administered for 3 days
by oral route in ovariectomized immature rats

| | Dose | Uterine weight gain |
|---|---|---|
| Treatment | (mg/kg/d) | (%) |
| 17α-Ethynylestradiol | 0.01 | 514% |
| Ethanolic extract of | | |
| Stem bark | 1000 | 7% |
| Twigs | 250 | 23% |
| Leaves | 250 | 44% |
| Seeds | 250 | 433% |
| n-Butanol soluble fraction | 250 | 43% |
| of ethanolic extract of stem bark | | |
| Aqueous extract of stem bark | 250 | 10% |

### Estrogen Antagonistic Activity

For evaluation of estrogen agonistic activity of ethanolic extract of stem bark, leaves and seed of Butea monosperma and fractions, bilaterally ovariectomized immature rats were treated with the test agents along with 0.01 g/kg daily dose of ethynylestradiol once daily for 3 consecutive days by the oral route. At autopsy 24 h after the last treatment, inhibition in ethynylestradiol induced uterine weight gain was determined. Ethanolic extract of stem bark of Butea monosperma at 1000 mg/kg daily dose produced 4% inhibition in 17α-ethynylestradiol induced uterine weight gain, as compared to 37% inhibition observed with 0.25 mg/kg daily dose of the antiestrogen raloxifene (Table 9).

TABLE 9
Estrogen antagonistic activity evaluation of ethanolic extract of
stem bark of Butea monosperma in ovariectomized immature rats

| | Daily dose | Inhibition in 17α-ethynylestradiol |
|---|---|---|
| Treatment | (mg/kg) | induced uterine weight gain |
| Ethanolic extract of | 1000 | 4% |
| stem bark of Butea | | |
| monosperma | | |
| Raloxifene | 0.25 | 37% |

B. Bioevaluation of Fractions of Ethanolic Extract of Butea monosperma

Using expression of alkaline phosphatase by primary osteoblast cell cultured for 48 h as parameter, promising osteoblast proliferative activity was localised in the n-butanol soluble fraction of the extract. Moderate activity was also observed in chloroform soluble fraction, while hexane and aqueous fractions were inactive (FIG. 10).

C. Bioevaluation of Compounds Isolated from Chloroform and n-butanol Soluble Fractions of Ethanolic Extract of Butea monosperma

Based on alkaline phosphatase expression and MTT assays for osteoblast proliferation and mineralisation in vitro, promising osteogenic activity was observed in six compounds nos. K051, K052, K054, K080, K082 (isolated from n-butanol soluble fraction) and K095 (isolated from chloroform soluble fraction) (FIGS. 11-15; Tables 10-11).

TABLE 10
Quantification of alkaline phosphatase activity in osteoblasts cultured in presence of varying concentration
of pure compounds isolated from active ethanolic extract of stem bark of Butea monosperma

| | Concentrations (M) | | | | | | | |
|---|---|---|---|---|---|---|---|---|
| Compounds | 10⁻¹¹ | 10⁻¹⁰ | 10⁻⁹ | 10⁻⁸ | 10⁻⁷ | 10⁻⁶ | 10⁻⁵ | Control |
| K052 | 3.17 ± 0.06 | 3.01 ± 0.26 | 3.07 ± 0.10 | 3.20 ± 0.06 | 3.05 ± 0.10 | 3.01 ± 0.09 | 2.29 ± 0.24 | 1.87 ± 0.20 |
| K080 | 3.38 ± 0.02 | 3.27 ± 0.08 | 3.33 ± 0.02 | 3.30 ± 0.01 | 3.46 ± 0.06 | 3.40 ± 0.02 | 3.45 ± 0.06 | 1.87 ± 0.20 |
| K095 | 3.51 ± 0.01 | 3.40 ± 0.07 | 3.54 ± 0.04 | 3.35 ± 0.10 | 3.50 ± 0.07 | 3.42 ± 0.07 | 3.28 ± 0.07 | 1.87 ± 0.20 |
| K051 | 3.63 ± 0.04 | 3.59 ± 0.08 | 3.50 ± 0.07 | 3.41 ± 0.12 | 3.23 ± 0.11 | 3.24 ± 0.09 | 3.26 ± 0.07 | 1.87 ± 0.20 |
| K054 | 3.01 ± 0.25 | 3.22 ± 0.12 | 3.32 ± 0.09 | 3.35 ± 0.03 | 3.32 ± 0.06 | 3.25 ± 0.07 | 2.65 ± 0.09 | 1.87 ± 0.20 |

### Values are Mean±SEM

### Evaluation of Alkaline Phosphatase Activity

p-Nitrophenol phosphate is a colorless substrate which is hydrolysed to colored p-nitrophenol by alkaline phosphatase enzyme. Rate of hydrolysis of p-nitrophenol phosphate is proportional to enzyme present in sample. For expression of alkaline phosphatase (ALP) activity, osteoblast cells (˜10⁴) plated on plastic cover slips (6 mm diameter) are incubated in the presence or absence of the test agent for 48 h, fixed in formalin and the alkaline phosphatase activity is displayed by incubation with ALP substrate solution (5 mg naphthol AS-MX phosphate, 0.25 ml ethylene glycol monomethyl ether, 10 mg Fast red TR, in 24 ml of 0.1M TBS, pH 9.5) for 1 h at room temperature. Findings reveal that all the five pure compounds nos. K051, K052, K054, K080 and K095 increased expression of alkaline phosphatase, a marker of osteoblast differentiation, in the concentration range of 10⁻¹¹ M to 10⁻⁵ M when compared to corresponding vehicle control group (FIG. 11, Table 10).

### MTT Assay for Cell Proliferation

This assay is based on the ability of viable cells to reduce tetrazolium salt to form formazone crystals by mitochondrial dehydrogenase enzyme. In MTT assay, osteoblasts are maintained in α-MEM medium supplemented with 10% FCS and 1% antibiotic solution in 96 well plate. When the cells attain 40% confluency, they are treated in presence or absence of test agents in 2% FCS supplemented media for 24 h. Twenty-four hours thereafter, MTT salt is added to the cells. After 4 h, the formazone crystals formed due to reduction of tetrazolium salt by mitochondrial dehydrogenase enzyme are dissolved in DMSO and readings taken at wavelength of 570 nm. All the five pure compounds nos. K051, K052, K054, K080 and K095 enhanced osteoblast cell proliferation after 24 h in concentration range of 10⁻¹¹ M to 10⁻⁵ M when compared to vehicle control group. Of these, compound K052 was found to be most potent followed by K080, K095, K051 and K054 (FIG. 12, Table 11).

TABLE 11
Osteoblast cell proliferation cultured in presence of varying concentration of pure compounds
isolated from active ethanolic extract of stem bark of Butea monosperma using MTT assay

| Compound | Concentration (M) | | | | | | | |
|---|---|---|---|---|---|---|---|---|
| no. | 10⁻¹¹ | 10⁻¹⁰ | 10⁻⁹ | 10⁻⁸ | 10⁻⁷ | 10⁻⁶ | 10⁻⁵ | Vehicle |
| K052 | 1.45 ± 0.23 | 1.43 ± 0.05 | 1.70 ± 0.08 | 1.73 ± 0.09 | 1.70 ± 0.15 | 1.86 ± 0.13 | 1.19 ± 0.01 | 0.60 ± 0.06 |
| K080 | 1.25 ± 0.09 | 1.35 ± 0.03 | 1.58 ± 0.09 | 1.47 ± 0.02 | 1.43 ± 0.09 | 1.55 ± 0.05 | 1.49 ± 0.03 | 0.60 ± 0.06 |
| K095 | 1.05 ± 0.03 | 1.38 ± 0.18 | 1.14 ± 0.19 | 1.24 ± 0.18 | 1.35 ± 0.16 | 1.47 ± 0.14 | 1.28 ± 0.08 | 0.60 ± 0.06 |
| K051 | 0.85 ± 0.05 | 0.92 ± 0.05 | 1.03 ± 0.03 | 1.32 ± 0.04 | 1.33 ± 0.01 | 1.34 ± 0.09 | 1.32 ± 0.05 | 0.60 ± 0.06 |
| K054 | 0.75 ± 0.06 | 0.88 ± 0.09 | 1.06 ± 0.04 | 1.24 ± 0.06 | 1.26 ± 0.07 | 1.24 ± 0.01 | 1.40 ± 0.02 | 0.60 ± 0.06 |

### Values are Mean±SEM

### Quantification of Mineralization

For quantification of mineralization which is measured with increased deposition of nascent calcium in osteoblast cells, cells were cultured in the presence of test compounds for 7 days and stained with Alizarin red. Alizarin red was extracted with acetic acid and the intensity of stain, which is directly proportion to the extent of mineralisation, is read at 405 nm. Result clearly demonstrated that when compared to vehicle control group, all the six pure compounds nos. K051, K052, K054, K080, K082 and K095 enhanced mineralisation as quantified by alizarin extraction method (FIG. 13).

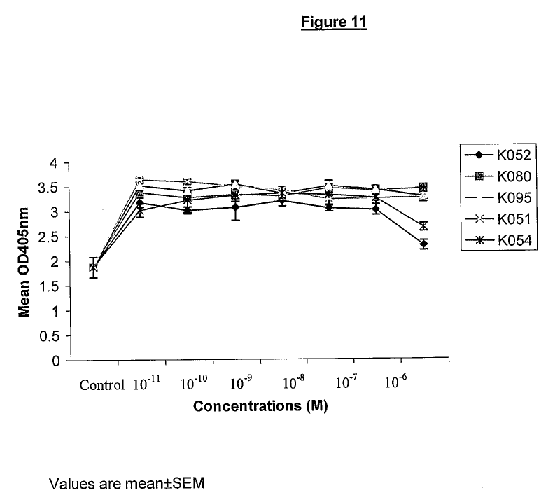

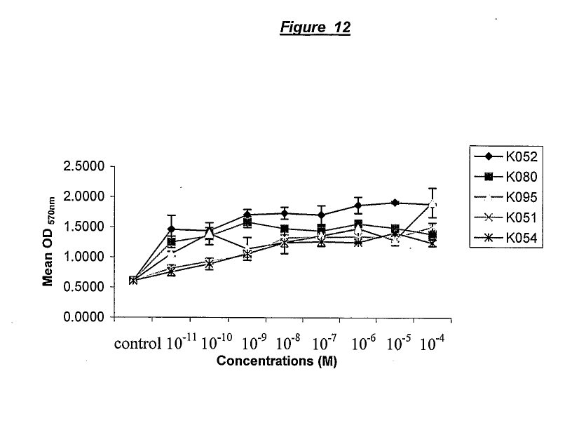

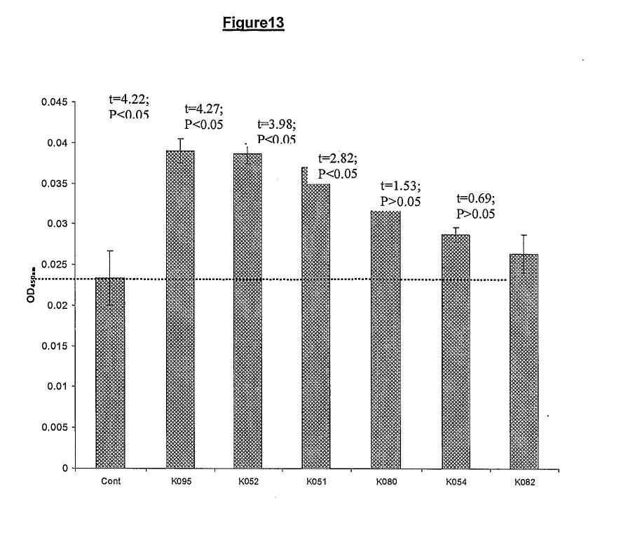

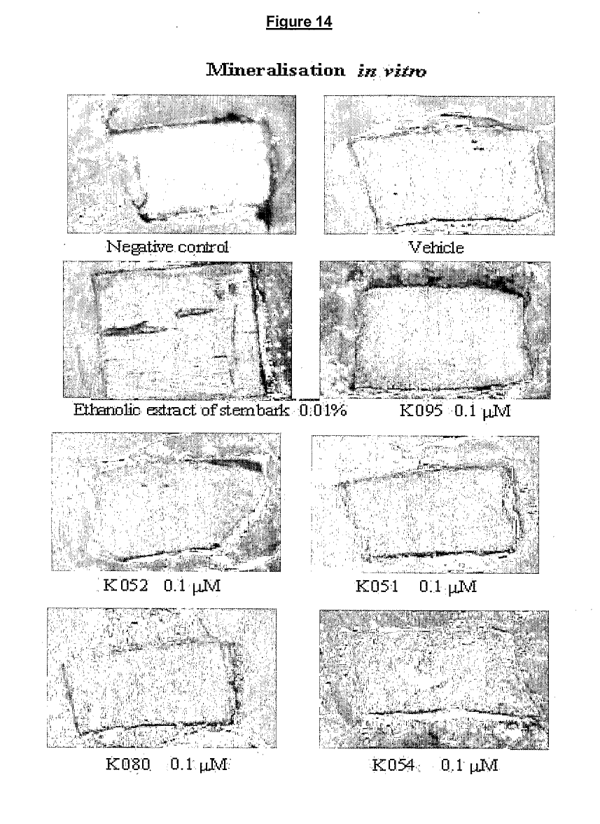

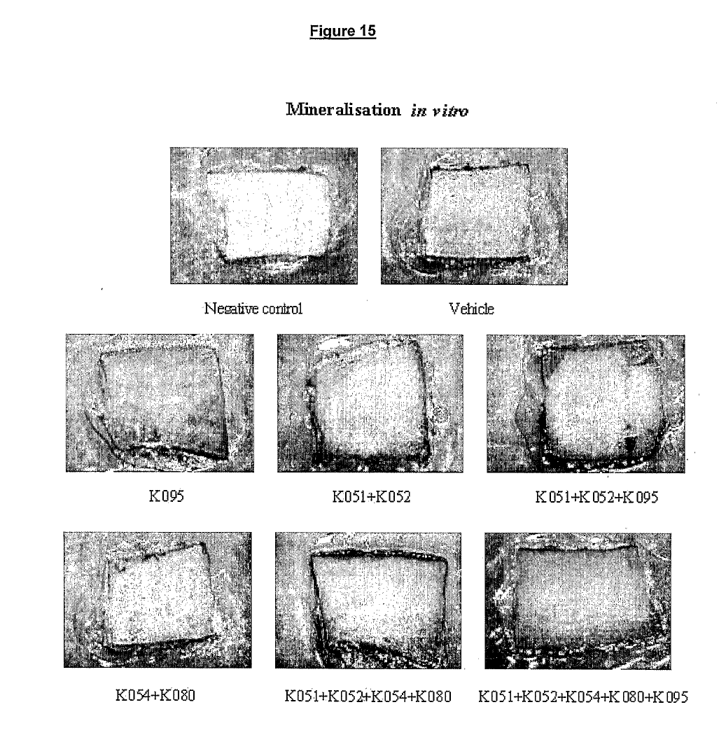

In case of osteoblasts seeded onto sterile bovine bone slices, the cells are cultured in 96-well plate in A-MEM supplemented with 10% fetal bovine serum, 2 mM glutamine, 100 U/ml penicillin-streptomycin, non-essential amino acid solution, sodium pyruvate, 10 mM p-glycerophosphate and 50 μg/ml ascorbic acid in the absence or presence of test compounds or their mixtures. Culture medium is changed every alternate day. At termination of the culture after 15 days, cells are washed twice with PBS, fixed in cold 70% ethanol for 1 h and washed once with water and stained with 40 mM Alizarin red (pH 4.7) for 30 min and then washed in PBS to remove excess staining. Results reveal that higher intensity of colour, demonstrating increased rate of new bone formation, was evident in cultures in the presence of all the five test compounds and their mixtures. K095 was found to be most potent (FIGS. 14 and 15).

1. A pharmaceutical composition for prevention or treatment of bone disorders comprising a therapeutically effective amount of one or more extract(s) or fraction(s) obtained from Butea species or compounds of formula 1 isolated therefrom or other natural sources or synthesized, their analogs or salts. 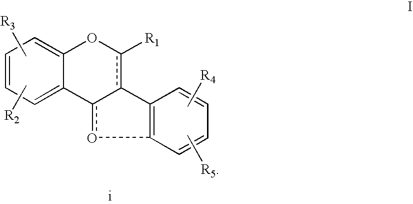
wherein the values of R₁, R₂, R₃, R₄, and R₅ in the compound of formula 1 are independently selected from the group consisting of hydrogen, methyl, hydroxy, and methoxy.

2. A pharmaceutical composition as claimed in claim 1 wherein compound(s) used are selected from the group consisting of the compounds represented by the formulas K0S1, K052, K054, K080, K082, and K095.

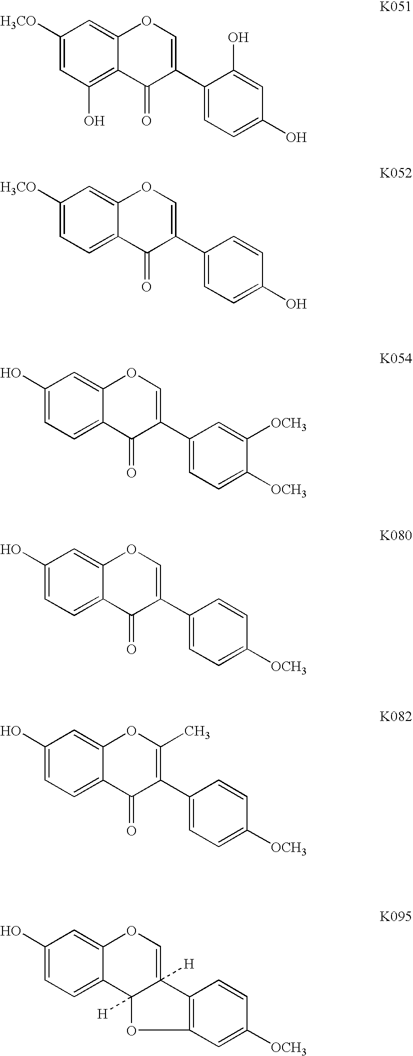

3. A pharmaceutical composition as claimed in claim 1 wherein the compounds are used either alone or in combination in the ratio ranging between 1 to 10 based on proportion, molar concentration or percent yield.

4. A pharmaceutical composition as claimed in claim 1 wherein the compounds K051 and K052 are used either alone or in combination based on molar concentration, percent yield, in equal or any proportions.

5. A pharmaceutical composition as claimed in claim 1 wherein the compounds K051, K052 and K095 are used either alone or in combination based on molar concentration, percent yield, in equal or any proportions.

6. A pharmaceutical composition as claimed in claim 1 wherein the compounds K054 and K080 are used either alone or in combination based on molar concentration, percent yield, in equal or any proportions.

7. A pharmaceutical composition as claimed in claim 1 wherein the compounds K051, K052, K054 and K080 are used either alone or in combination based on molar concentration, percent yield, in equal or any proportions.

8. A pharmaceutical composition as claimed in claim 1 wherein the compounds K061, K052, K054, K080 and K095 are used either alone or in combination based on molar concentration, percent yield, in equal or any proportions.

9. A pharmaceutical composition as claimed in claim 1 wherein the compounds K051, K052, K054, K080, K082 and K095 are used either alone or in combination based on molar concentration, percent yield, in equal or any proportions.

10. A pharmaceutical composition as claimed in claim 1 wherein the concentration of each compound used either alone or in combination is 0.1 μM.

11. A composition as claimed in claim 1 wherein the pharmaceutical composition further comprises a diluent selected from the group consisting of lactose, mannitol, sorbitol, microcrystalline cellulose, sucrose, sodium citrate, dicalcium phosphate, and combinations thereof.

12. A composition as claimed in claim 1 wherein the pharmaceutical composition further comprises one or more pharmaceutical acceptable excipients selected from the group consisting of:
(a) a diluent selected from the group consisting of lactose, mannitol, sorbitol, microcrystalline cellulose, sucrose, sodium citrate, dicalcium phosphate and combinations thereof;
(b) a binder selected from the group consisting of gum tragacanth, gum acacia, methyl cellulose, gelatin, polyvinyl pyrrol idone, starch and combinations thereof;
(c) a disintegrating agent selected from the group consisting of agar-agar, calcium carbonate, sodium carbonate, silicates, alginic acid, corn starch, potato tapioca starch, primogel and combinations thereof;
(d) a lubricant selected from the group consisting of magnesium stearate, calcium stearate or steorotes, talc, solid polyethylene glycols, sodium lauryl sulphate and combinations thereof;
(e) a glidant selected from the group consisting of colloidal silicon dioxide;
(f) a sweetening agent selected from the group consisting of sucrose, saccharin and combinations thereof;
(g) a flavoring agent selected from the group consisting of peppermint, methyl salicylate, orange flavor, vanilla flavor, and combinations thereof;
(h) wetting agents selected from the group consisting of cetyl alcohol, glyceryl monostearate and combinations thereof;
(i) absorbents selected from the group consisting of kaolin, bentonite clay and combinations thereof; and
solution retarding agents selected from the group consisting of wax, paraffin and combinations thereof.

13. A composition as claimed in claim 1 wherein the effective dose of the composition is ranging between 0.1 to 5000 mg per kg body weight, administered daily, bi-weekly, weekly or in more divided doses.

14. A composition as claimed in claim 1 wherein the bone disorders being prevented or treated are selected from the group consisting of osteoporosis, bone loss, bone formation, bone fracture healing, attainment of higher peak bone mass when administered during the period of growth, and promotion of new bone formation in vitro/in vivo.

15. A composition as claimed in claim 1 wherein an ethanolic extract of stem bark showed greater intensity in alkaline phosphatase staining when compared to vehicle control osteoblast cell cultures at time intervals of 24 h and 48 h.

16. A composition as claimed in claim 1 wherein an ethanolic extract of stem bark exhibiting total alkaline phosphate activity higher by 58% as compared to 28% increase in enzyme activity in presence of sodium β-glycerophosphate per se treated bones.

17. A composition as claimed in claim 1 wherein an ethanolic extract of stem bark induced marked proliferation of primary osteoblasts in culture when compared to corresponding vehicle control group at a concentration of 0.05% and 0.1% wherein the percent viable cells are 330% and 361%, respectively in comparison to that of vehicle control group taken as 100%.

18. A composition as claimed in claim 1 wherein an ethanolic extract of stem bark at its osteogenic concentrations (0.05% and 0.1%) did not exhibit any proliferative effect on Ishikawa (human uterine glandular epithelial carcinoma) or MCF-7 (human cancer breast) cell lines.

19. A composition as claimed in claim 1 wherein an osteoblast specific proliferation effect of the extract demonstrates lack of any estrogen agonistic action of the extract at the endometrial and breast levels.

20. A composition as claimed in claim 1 wherein an ethanolic extract of stem bark exhibiting more than 2.5-fold increase in expression of collagen-I (a marker of osteoblast proliferation and differentiation) in calvaria of 21-day old rats 72 h after single 1000 mg/kg oral dose.

21. A composition as claimed in claim 1 wherein an ethanolic extract of stem bark exhibiting more than 5 fold increase in the expression of osteocalcin, a marker of extracellular matrix maturation in the calvaria of 21-day old rats 72 h after single 1000 mg/kg oral dose.

22. A composition as claimed in claim 1 wherein an ethanolic extract of stem bark exhibiting no effect of the treatment on expression of glyceraldehyde 3-phosphate dehydrogenase (GAPOH)₁ a house-keeping gene.

23. A composition as claimed in claim 1 wherein an ethanolic extract of stem bark exhibiting increased rate of mineralization in vitro wherein higher intensity of alizarin red staining depicting increased deposition of nascent calcium in osteoblasts at both 24 h and 48 h with respect to corresponding vehicle controls.

24. A composition as claimed in claim 1 wherein an ethanolic extract of stem bark exhibiting increased rate of mineralization in osteoblasts cultured for 7 days in vitro with respect to corresponding sodium β-glycerophosphate per se treated or vehicle control cultures, at a concentration of 0.1%.

25. A composition as claimed in claim 1 wherein increased incidence of mineralised nodules in osteoblast cell cultures treated with ethanolic extract of stem bark for 15 and 25 days demonstrating increased rate of new bone formation.

26. A composition as claimed in claim 1 wherein higher intensity of alizarin staining was evident in long term osteoblast cell cultured on sterile bovine bone slices in the presence of ethanolic extract of stem bark for 18 and 30 days demonstrating increased rate of new bone formation.

27. A composition as claimed in claim 1 wherein an ethanolic extract of stem bark on employing its effective osteogenic concentration, exhibited positive response by promoting bone formation as evidenced by T/C ratio of ≦0.5 in chick fetal bone culture assay.

28. A composition as claimed in claim 1 wherein an ethanolic extract on employing or administering its effective osteogenic concentration did not inhibit PTH induced resorption of ⁴⁶Ca from chick fetal bones in culture with T/C ratio of 1.34, in comparison to T/C ratio of 0.66 and 0.37 in presence of 100 μM concentration of raloxifene and estradiol 17β.

29. A composition as claimed in claim 1 wherein an ethanolic extract on oral administration at 1000 mg/kg daily dose for 30 consecutive days markedly increased (5% to 65%) bone mineral density (BMO) of all regions of Lumbar spine, femur and tibia bones of immature female Sprague-Dawley rats when compared with that of corresponding vehicle control group.

30. A composition as claimed in claim 1 wherein the bones of immature rats treated with the extract also exhibiting higher mechanical strength as evidenced by greater force required to break the femur bone using three-pointing bending test for fracture and for compression of the Lumbar-3 vertebra using TK252C Muromachi Bone Strength Tester.

31. A composition as claimed in claim 1 wherein an ethanolic extract on oral administration at 1000 mg/kg daily dose for 30 consecutive days markedly increased new bone formation as evidenced by double labeling technique involving administration of calcium seeking agents tetracycline at the time of start of treatment and calcein at the time of completion of treatment, sectioning of the undecalcified bones and visualisation of tetracycline label under UV light and calcein under orange filter.

32. A composition as claimed in claim 1 wherein an ethanolic extract of stem bark is devoid of any estrogen agonistic activity at the uterine level when administered at 1000 mg/kg daily dose for 3 days in ovariectomized immature rats and 30 days in intact immature rats.

33. A composition as claimed in claim 1 wherein there was no effect of an ethanolic extract of stem bark on rate of age-related increase in body weight or uterine weight in immature rats.

34. The composition as claimed in claim 1 wherein a composition comprising an ethanolic extract of seeds exhibited potent estrogen agonistic activity as evidenced by marked (433%) an increase in uterine fresh weight in immature rat bioassay, comparable to that induced by 0.01 g/kg daily dose of ethynylestradiol.

35. A composition as claimed in claim 1 wherein an ethanolic extract of stem bark at 1000 mg/kg daily dose administered for 3 consecutive days to ovariectomized immature rats produced 4% inhibition in 17α-ethynylestradiol (0.01 mg/kg/day) induced uterine weight gain, as compared to 37% inhibition observed with 0.25 mg/kg daily dose of the antiestrogen raloxifene.

36. A composition as claimed in claim 1 wherein the Butea species is selected from the group consisting of Butea monosperma, Butee parviflora, Butea minor and Butea superba.

37. A composition as claimed in claim 36 wherein the Butea species is Butea monosperma and wherein the composition is derived from plant parts selected from the group consisting of stem bark, twigs, leaves, flowers, and seeds.

38. A composition as claimed in claim 1 wherein the bioactlve extract/fraction is selected from the group consisting of alcoholic extract, chloroform soluble fraction, and n-butanol soluble fraction.

39. A composition as claimed in claim 1 wherein n-butanol soluble and chloroform soluble fractions of the ethanolic extract of stem bark showed greater intensity in alkaline phosphatase staining when compared to corresponding vehicle (ethanol:DMSO, 50:50, v/v) control osteoblast cell cultures at 48 h.

40. A composition a claimed in claim 1 wherein compounds K051, K052, K054, K080 and K095 increased expression of alkaline phosphatase (a marker of osteoblast differentiation), in osteoblasts plated on plastic cover slips (6 mm diameter) and incubated for 48 h in the concentration range of 10⁻¹¹ M to 10⁻⁵ M when compared to corresponding vehicle control group.

41. A composition as claimed in claim 1 wherein compounds K051, K052, K054, K080 and K095 enhanced osteoblast cell proliferation after 24 h in concentration range of 10⁻¹¹ M to 10⁻⁶ M when compared to vehicle control group in MTT assay.

42. A composition as claimed in claim 1 wherein compounds nos. K051, K052, K054, K080, K082 and K095 enhanced mineralisation as evidenced by increased deposition of nascent calcium in osteoblast cells cultured for 7 days and quantified by alizarin extraction method.

43. A composition as claimed in claim 1 wherein the compounds K051, K052, K054, K080, K082 and K095 used either alone or in combination increased intensity of alizarin red staining, demonstrating increased rate of new bone formation, in osteoblasts cultured on sterile bovine bone slices in 96-well plate for 15 days.

44. A process for the preparation of bioactive fraction from Butea species as claimed in claim 1, wherein the process comprises:
(a) soaking the powdered plant parts in alcoholic solvent and removing and concentrating the solvent by conventional methods to obtain alcoholic extract;
(b) triturating the alcoholic extract obtained from step (a) with hexane to obtain the hexane soluble fraction and hexane insoluble fraction,
(c) triturating the hexane insoluble fraction with chloroform to obtain chloroform soluble fraction and chloroform insoluble fraction,
(d) subjecting the chloroform soluble fraction to repeated chromatography to obtain compounds K084, K090, K095, K103, K10S, K113, K115,
(e) partitioning the chloroform insoluble fraction with n-butanol and water to obtain n-butanol soluble fraction and aqueous fraction, and
(f) subjecting the n-butanol soluble fraction to repeated chromatography to obtain compounds K010, K039, K040, K051, K052, K053, K054, K064, K080, K082, K098, and K111.

45. A process as claimed in claim 44 wherein the alcohol used for extraction is selected from the group consisting of methanol, ethanol, propanol and mixtures thereof.

46. A process as claimed in claim 44 wherein the chromatographic method used for isolation of compounds is selected from the group consisting of column, flash, medium pressure and HPLC.

47. A process as claimed in claim 44 wherein the compounds may be converted to their pharmaceutically acceptable salts, wherein the salts are selected from the group consisting of hydrochloride, formate, acetate, phenyl acetate, trifluroacetate, acrylate, ascorbate, benzoate, chlorobenzoates, bromobezoates, iodobenzoates, nitrobenzoates, hydroxybenzoates, alkylbenzoates, alkyloxybenzoates, alkoxycarbonylbenzoates, naphthalene-2 benzoate, butyrates, phenylbutyrates, hydroxybutyrates, caprate, capryiate, cinnamate, mandelate, mesylate, citrate, tartarate, fumerate, heptanoate, hippurate, lactate, malate, maleate, malonate, nicotinate, isonicotinate, oxalate, phthalate, terephthalate, phosphate, monohydrogan phosphate, dihydrogen phosphate, metaphosphate, pyrophosphate, propioiate, propionate, phenylprapionate, salicylate, sebacte, succinate, suberate, sulphate, bisulphate, pyrosulphate, sulphite, bisulphate, sulphonate, benzene sulphonate, bromobenzene sulphonates, chlorobenzene sulphonates, ethane sulphonates, methane sulphonates, naphthalene sulphonates, and toluene sulphonates.

48. A method for prevention or treatment of bone disorders wherein said method comprising the steps of administering to a subject in need thereof a pharmaceutical composition as claimed in claim 1.

49. A method as claimed in claim 48 wherein the composition is administered by the route selected from the group consisting of oral, percutaneous, intramuscular, intraperitoneal, intravenous, and local.

50. A method as claimed in claim 48 wherein the composition is used in a dose ranging between 1 to 5000 mg/kg body weight.

51. A method as claimed in claim 48 wherein the composition is used in a form selected from the group consisting of tablet, syrup, powder, capsule, suspension, solution, ointment, and mixture.
